(19) United States
(12) Patent Application Publication
(10) Pub. No.: US 2022/0267733 A1
(43) Pub. Date: Aug. 25, 2022

(54) METHODS OF PRODUCING HAEMOGENIC PROGENITOR CELLS FROM PLURIPOTENT STEM CELLS

(71) Applicant: Adaptimmune Limited, Abingdon Oxfordshire (GB)

(72) Inventors: Cheng Tao Yang, Abingdon Oxfordshire (GB); Lee Carpenter, Abingdon Oxfordshire (GB)

(21) Appl. No.: 17/630,882

(22) PCT Filed: Aug. 20, 2020
(86) PCT No.: PCT/EP2020/073396

§ 371 Date: Jan. 27, 2022

(30) Foreign Application Priority Data
Aug. 20, 2019 (GB) 1911957.7

(51) Int. Cl. C12N 5/0789; C12N 5/0783; C12N 5/10

(52) U.S. Cl. CPC C12N 5/0647; C12N 5/0636; C12N 5/10; C12N 2501/115; C12N 2501/125; C12N 2501/145; C12N 2501/15; C12N 2501/16; C12N 2501/2306; C12N 2501/2303; C12N 2501/2307; C12N 2501/515

(57) ABSTRACT

This invention relates to the production of a population of haemogenic progenitor cells by (i) differentiating a population of induced pluri potent stem cells (IPSCs) into mesoderm cells and; (II) differentiating the mesoderm cells to produce a population of haemogenic progenitor cells. Steps (i) and (ii) are performed without purification or isolation of cells in the population. In addition, the haemogenic progenitor cells may be produced without the use of serum or stromal co-culture. Methods of the invention may be useful for example, in the production of clinical grade blood cells, such as T cells, for use in immunotherapy.

### FIELD

This invention relates to the production of haemogenic progenitor cells, such as haemogenic endothelial cells (HECs) and haematopoietic progenitor cells (HPCs), for example for use in generating T cells for immunotherapy.

### BACKGROUND

Immunotherapeutics are poised to transform the cancer treatment landscape with the promise of long-term survival (McDermott et al., Cancer Treat Rev. 2014 October; 40(9): 1056-64). There is a clear unmet medical need for new immunomodulatory drugs to expand patient population and range of tumor types. In addition, new agents are needed to enhance the magnitude and duration of anti-tumor responses. The development of these agents has been possible because of the in-depth understanding of the basic principles controlling T-cell immunity over the last two decades (Sharma and Allison, Cell. 2015. Apr 9; 161(2): 205-14). This typically requires tumor specific CD4+ and CD8+ T-cells recognising tumor-associated peptide antigens presented by MHC molecules. Different vaccination strategies and adoptive transfer of ex vivo expanded tumor infiltrated lymphocytes have in some cases demonstrated the ability of tumor specific T-cells to treat late stage cancer (Rosenberg et al., Nat Med. 2004 September; 10(9): 909-15).

However, current adoptive T cell therapies are limited by a lack of suitable patient and tumor-specific T cells and there is a need for therapeutically sufficient and functional antigen-specific T cells for effective use in immunotherapy.

### SUMMARY

The present inventors have unexpectedly found that haemogenic progenitor cells, such as haemogenic endothelial cells (HECs) or haematopoietic progenitor cells (HPCs), that are capable of differentiation into T cells, may be produced from induced pluripotent stem cells (iPSCs) in a single culture vessel without intermediate purification or isolation steps. In addition, the haemogenic progenitor cells may be produced without the use of serum or stromal co-culture. This may be useful for example in the production of clinical grade blood cells, such as T cells, for use in immunotherapy.

A first aspect of the invention provides a method of producing a population of haemogenic progenitor cells comprising;
(i) differentiating a population of induced pluripotent stem cells (iPSCs) into mesoderm cells and;
(ii) differentiating the mesoderm cells to produce a population of haemogenic progenitor cells
wherein steps (i) and (ii) are performed without purification or isolation of cells in the population.

Haemogenic progenitor cells may include haemogenic endothelial cells (HECs). A method of the first aspect may comprise;
(i) differentiating a population of induced pluripotent stem cells (iPSCs) into mesoderm cells and;
(ii) differentiating the mesoderm cells to produce a population of HECs, wherein steps (i) and (ii) are performed without purification or isolation of cells in the population.

Haemogenic progenitor cells may include haematopoietic progenitor cells (HPCs). A method of the first aspect may comprise;
(i) differentiating a population of induced pluripotent stem cells (iPSCs) into mesoderm cells
(ii) differentiating the mesoderm cells into HECs, and
(iii) differentiating the HECs into a population of haematopoietic progenitor cells (HPCs),
wherein steps (i), (ii) and (iii) are performed without purification or isolation of cells in the population.

Preferably, the iPSCs are differentiated into haemogenic progenitor cells, such as HECs or HPCs, in steps (i), (ii) and (iii) above in defined culture media in the absence of feeder cells, stromal cells, serum or other undefined media supplements.

In some preferred embodiments, HPCs as described herein may be used in the production of T cells. A method of the first aspect may further comprise;
(iv) differentiating the population of HPCs into T cell progenitor cells; and
(v) maturing the progenitor T cells to produce a population of DP CD4+CD8+T cells.

A method of the first aspect may further comprise;
(vi) activating and expanding the DP CD4+ CD8+ T cells to produce a population of SP CD8+ T cells or a population of SP CD4+ T cells.

In other embodiments, HPCs are described herein may be used in the production of NK cells. A method of the first aspect may further comprise;
(iv) differentiating the population of HPCs into NK cells.

In other embodiments, HPCs are described herein may be used in the production of B cells. A method of the first aspect may further comprise;
(iv) differentiating the population of HPCs into B cells.

These and other aspects and embodiments of the invention are described in more detail below.

### BRIEF DESCRIPTION OF FIGURES

FIG. 1 shows a schematic view of an example of a six-stage method for generating T cells from iPSCs.

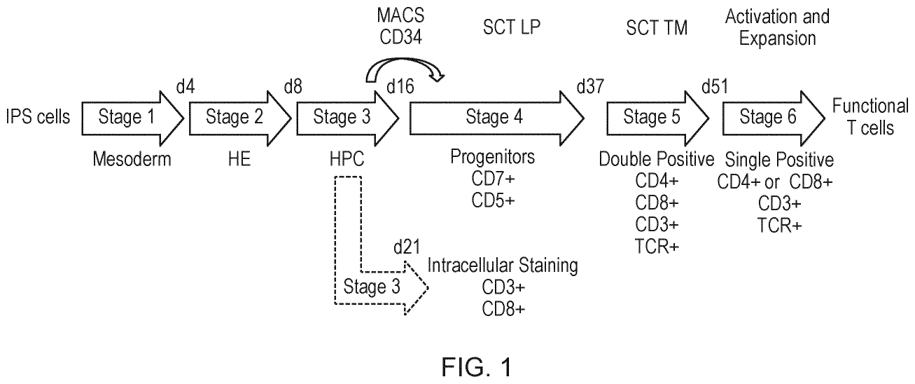

### DETAILED DESCRIPTION

This invention relates to the finding that haemogenic progenitor cells, such as haemogenic endothelial cells (HECs) and haematopoietic progenitor cells (HPCs), can be produced from induced pluripotent stem cells (iPSCs) without the use of purification steps, such as cell sorting or gating. This allows the production of the haemogenic progenitor cells in a single culture vessel.

Preferably, the iPSCs are differentiated into haemogenic progenitor cells, such as HECs or HPCs, in defined culture media in the absence of feeder cells, stromal cells, such as OP9-DI4 stromal cells, serum or other undefined media supplements. This allows the production of clinical grade cell products and HECs and HPCs produced as described herein may be useful, for example, in the production of T cells for use in immunotherapy.

Induced pluripotent stem cells (iPSCs) are pluripotent cells which are derived from non-pluripotent, fully differentiated donor or antecedent cells. iPSCs are capable of self-renewal in vitro and exhibit an undifferentiated phenotype and are potentially capable of differentiating into any foetal or adult cell type of any of the three germ layers (endoderm, mesoderm and ectoderm). The population of iPSCs may be clonal i.e. genetically identical cells descended from a single common ancestor cell.

iPSCs may express one or more of the following pluripotency associated markers: POU5f1 (Oct4), Sox2, Alkaline Phosphatase, SSEA-3, Nanog, SSEA-4, Tra-1-60, KLF4 and c-myc, preferably one or more of POU5f1, NANOG and SOX2. An iPSC may lack markers associated with specific differentiative fates, such as Brachyury, Sox17, FoxA2, αFP, Sox1, NCAM, GATA6, GATA4, Hand1 and CDX2. In particular, an iPSC may lack markers associated with endodermal fates.

Preferably, the iPSCs are human IPSCs (hiPSCs).

In some embodiments, iPSCs may be gene edited, for example to inactivate or delete HLA genes or other genes associated with immunogenicity or GVHD, or may be gene edited to include nucleic acid encoding an exogenous antigen receptor such as for example a TCR, CAR or NKCR.

IPSCs may be derived or reprogramed from donor cells, which may be somatic cells or other antecedent cells obtained from a source, such as a donor individual. The donor cells may be mammalian, preferably human cells. Suitable donor cells include adult fibroblasts and blood cells, for example peripheral blood cells, such as HPCs, mononuclear cells, CD34+ cord blood cells or T-cells.

Suitable donor cells for reprogramming into iPSCs as described herein may be obtained from a donor individual. In some embodiments, the donor individual may be the same person as the recipient individual to whom the T cells will be administered following production as described herein (autologous treatment). In other embodiments, the donor individual may be a different person to the recipient individual to whom the T cells will be administered following production as described herein (allogeneic treatment). For example, the donor individual may be a healthy individual who is human leukocyte antigen (HLA) matched (either before or after donation) with a recipient individual suffering from cancer. In other embodiments, the donor individual may not be HLA matched with the recipient individual. Preferably, the donor individual may be a neonate (new-born), for example the donor cells may be obtained from a sample of umbilical cord blood.

Suitable donor individuals are preferably free of communicable viral (e.g. HIV, HPV, CMV) and adventitious agents (e.g. bacteria, mycoplasma), and free of known genetic abnormalities.

In some embodiments, a population of peripheral blood cells, such as HPCs, for reprogramming may be isolated from a blood sample, preferably an umbilical cord sample, obtained from the donor individual. Suitable methods for the isolation of HPCs and other peripheral blood cells, are well-known in the art and include, for example magnetic activated cell sorting (see, for example, Gaudernack et al 1986 J Immunol Methods 90 179), fluorescent activated cell sorting (FACS: see for example, Rheinherz et al (1979) PNAS 76 4061), and cell panning (see for example, Lum et al (1982) Cell Immunol 72 122). HPCs may be identified in a sample of blood cells by expression of CD34. In other embodiments, a population of fibroblasts for reprogramming may be isolated from a skin biopsy following dispersal using collagenase or trypsin and out-growth in appropriate cell culture conditions.

In some embodiments, IPSCs may be derived from antigen-specific T cells. For example, the T cells may comprise nucleic acid encoding αβ TCRs that bind to an antigen, such as a tumor antigen, displayed in complex with a class 1 MHC. Antigen-specific T cells for use in the generation of iPSCs may be obtained by screening a diverse population of T cells with peptide epitopes from the target antigen displayed on a class I or II MHC molecule on the surface of an antigen presenting cell, such as a dendritic cell, or by isolating from a tumour sample from a cancer patient.

Donor cells are typically reprogrammed into iPSCs by the introduction of reprogramming factors, such as Oct4, Sox2 and Klf4 into the cell. The reprogramming factors may be proteins or encoding nucleic acids and may be introduced into the differentiated cells by any suitable technique, including plasmid, transposon or more preferably, viral transfection or direct protein delivery. Other reprogramming factors, for example Klf genes, such as Klf-1, -2, -4 and -5; Myc genes such as C-myc, L-myc and N-myc; Nanog; SV40 Large T antigen; Lin28; and short hairpins (shRNA) targeting genes such as p53, may also be introduced into the cell to increase induction efficiency. Following introduction of the reprogramming factors, the donor cells may be cultured. Cells expressing pluripotency markers may be isolated and/or purified to produce a population of iPSCs. Techniques for the production of iPSCs are well-known in the art (Yamanaka et al Nature 2007; 448:313-7; Yamanaka 6 2007 Jun. 7; 1(1):39-49; Kim et al Nature. 2008 Jul. 31; 454(7204):646-50; Takahashi Cell. 2007 Nov. 30; 131(5):861-72. Park et al Nature. 2008 Jan. 10; 451(7175):141-6; Kimet et al Cell Stem Cell. 2009 Jun. 5; 4(6):472-6; Vallier, L., et al. Stem Cells, 2009. 9999(999A): p. N/A; Baghbaderani et al 2016; Stem Cell Rev. 2016 August; 12(4):394-420; Baghbaderani et al. (2015) Stem Cell Reports, 5(4), 647-659).

Conventional techniques may be employed for the culture and maintenance of iPSCs (Vallier, L. et al Dev. Biol. 275, 403-421 (2004), Cowan, C.A. et al. N. Engl. J. Med. 350, 1353-1356 (2004), Joannides, A. et al. Stem Cells 24, 230-235 (2006) Klimanskaya, I. et al. Lancet 365, 1636-1641 (2005), Ludwig, T.E. et al. Nat. Biotechnol. 24, 185-187 (2006)). IPSCs for use in the present methods may be grown in defined conditions or on feeder cells. For example, iPSCs may be conventionally cultured in a culture dish on a layer of feeder cells, such as irradiated mouse embryonic fibroblasts (MEF), at an appropriate density (e.g. 10⁵ to 10⁶ cells/60mm dish), or on an appropriate substrate, in a feeder conditioned or defined iPSC maintenance medium. iPSCs for use in the present methods may be passaged by enzymatic or mechanical means. In some embodiments, iPSCs may be passaged on Matrigel™ or an ECM protein, such as vibronectin, in an iPSC maintenance medium, such as mTeSR™1 or TeSR™2 (StemCell Technologies) or E8 flex (Life Thermo) culture medium.

iPSCs may be differentiated into HECs using a two-step process that includes a mesoderm stage. For example, a method may comprise;
(i) differentiating the population of iPSCs into mesoderm cells, and
(ii) differentiating the mesoderm cells into HECs.

Preferably, the HECs are further differentiated into HPCs. For example, a method may further comprise;
(iii) differentiating the HECs into a population of HPCs.

Steps (i), (ii) and (iii) are all performed without purifying any cells or subpopulations from the population of cells in order to produce the HPCs with T cell potential. Steps (i), (ii) and (iii) may be performed without use of serum and stromal cells, such as OP9-D14 stromal cells. For example, the population of cells may remain in the same culture vessel and subjected to only to changes of culture medium in order to effect differentiation steps (i), (ii) and (iii), as described herein.

Differentiation and maturation of the cell populations in the steps of the methods described herein is induced by culturing the cells in a culture medium supplemented with a set of differentiation factors. The set of differentiation factors that is listed for each culture medium is preferably exhaustive and medium may be devoid of other differentiation factors. In preferred embodiments, the culture media are chemically defined media. For example, a culture medium may consist of a chemically defined nutrient medium that is supplemented with an effective amount of one or more differentiation factors, as described below. A chemically defined nutrient medium may comprise a basal medium that is supplemented with one or more serum-free culture medium supplements.

Differentiation factors are factors which modulate, for example promote or inhibit, a signalling pathway which mediates differentiation in a mammalian cell. Differentiation factors may include growth factors, cytokines and small molecules which modulate one or more of the Activin/Nodal, FGF, Wnt or BMP signalling pathways. Examples of differentiation factors include Activin/Nodal, FGFs, BMPs, retinoic acid, vascular endothelial growth factor (VEGF), stem cell factor (SCF), TGF ligands, GDFs, LIF, Interleukins, GSK-3 inhibitors and phosphatidylinositol 3-kinase (PI3K) inhibitors.

Differentiation factors which are used in one or more of the media described herein include TGFβ ligands, such as activin, fibroblast growth factor (FGF), bone morphogenetic protein (BMP), stem cell factor (SCF), vascular endothelial growth factor (VEGF), GSK-3 inhibitors (such as CHIR-99021), interleukins, and hormones, such as IGF-1 and angiotensin II. A differentiation factor may be present in a medium described herein in an amount that is effective to modulate a signalling pathway in cells cultured in the medium.

In some embodiments, a differentiation factor listed above or below may be replaced in a culture medium by a factor that has the same effect (i.e. stimulation or inhibition) on the same signalling pathway. Suitable factors are known in the art and include proteins, nucleic acids, antibodies and small molecules.

The extent of differentiation of the cell population during each step may be determined by monitoring and/or detecting the expression of one or more cell markers in the population of differentiating cells. For example, an increase in the expression of markers characteristic of the more differentiated cell type or a decrease in the expression of markers characteristic of the less differentiated cell type may be determined. The expression of cell markers may be determined by any suitable technique, including immunocytochemistry, immunofluorescence, RT-PCR, immunoblotting, fluorescence activated cell sorting (FACS), and enzymatic analysis. In preferred embodiments, a cell may be said to express a marker if the marker is detectable on the cell surface. For example, a cell which is stated herein not to express a marker may display active transcription and intracellular expression of the marker gene but detectable levels of the marker may not be present on the surface of the cell.

A population of partially differentiated cells, for example cells other than functional T cells, for example mesodermal cells, HECs (i.e. HE cells), HHPC, T cell progenitors or DP T cells, that is produced by a step in the methods described herein may be cultured, maintained or expanded before the next differentiation step. Partially differentiated cells may be expanded by any convenient technique.

Cells may be cultured in a monolayer, in the absence of feeder cells, on a surface or substrate coated with extracellular matrix protein, such as fibronectin, laminin or collagen. Suitable techniques for cell culture are well-known in the art (see, for example, Basic Cell Culture Protocols, C. Helgason, Humana Press Inc. U.S. (15 Oct. 2004) ISBN: 1588295451; Human Cell Culture Protocols (Methods in Molecular

Medicine S.) Humana Press Inc., U.S. (9 Dec. 2004) ISBN: 1588292223; Culture of Animal Cells: A Manual of Basic Technique, R. Freshney, John Wiley & Sons Inc (2 Aug. 2005) ISBN: 0471453293, Ho WY et al J Immunol Methods. (2006) 310:40-52, Handbook of Stem Cells (ed. R. Lanza) ISBN: 0124366430) Basic Cell Culture Protocols' by J. Pollard and J. M. Walker (1997), ‘Mammalian Cell Culture: Essential Techniques’ by A. Doyle and J. B. Griffiths (1997), ‘Human Embryonic Stem Cells’ by A. Chiu and M. Rao (2003), Stem Cells: From Bench to Bedside' by A. Bongso (2005), Peterson & Loring (2012)Human Stem Cell Manual: A Laboratory Guide Academic Press and ‘Human Embryonic Stem Cell Protocols’ by K. Turksen (2006). Media and ingredients thereof may be obtained from commercial sources (e.g. Gibco, Roche, Sigma, Europa bioproducts, R&D Systems). Standard mammalian cell culture conditions may be employed for the above culture steps, for example 37° C., 5% or 21% Oxygen, 5% Carbon Dioxide. Media is preferably changed every two days and cells allowed to settle by gravity.

Cells may be cultured in a culture vessel. Suitable cell culture vessels are well-known in the art and include culture plates, dishes, flasks, bioreactors, and multi-well plates, for example 6-well, 12-well or 96-well plates.

The culture vessels are preferably treated for tissue culture, for example by coating one or more surfaces of the vessel with an extracellular matrix protein, such as fibronectin, laminin or collagen. Culture vessels may be treated for tissue culture using standard techniques, for example by incubating with a coating solution as described herein, or may be obtained from pre-treated from commercial suppliers.

In a first stage, iPSCs may be differentiated into mesoderm cells by culturing the population of iPSCs under suitable conditions to promote mesodermal differentiation. For example, the iPSCs cells may be cultured sequentially in first, second and third mesoderm induction media to induce differentiation into mesoderm cells.

A suitable first mesoderm induction medium may stimulate SMAD2 and SMAD3 and/or SMAD2 and SMAD3 mediated signalling pathways. For example, the first mesoderm induction medium may comprise activin.

A suitable second mesoderm induction medium may (i) stimulate SMAD1, SMAD2, SMAD3, SMAD5 and SMAD9 and/or SMAD1, SMAD2, SMAD3, SMAD5 and SMAD9 mediated signalling pathways and (ii) have fibroblast growth factor (FGF) activity. For example, the second mesoderm induction medium may comprise activin, preferably activin A, BMP, preferably BMP4 and FGF, preferably bFGF.

A suitable third mesoderm induction medium may (i) stimulate SMAD1, SMAD2, SMAD3, SMAD5 and SMAD9 and/or SMAD1, SMAD2, SMAD3, SMAD5 and SMAD9 mediated signalling pathways (ii) have fibroblast growth factor (FGF) activity and (iii) inhibit glycogen synthase kinase 3β. For example, the third mesoderm induction medium may comprise activin, preferably activin A, BMP, preferably BMP4, FGF, preferably bFGF, and a GSK3 inhibitor, preferably CHIR99021.

The first, second and third mesoderm induction media may be devoid of differentiation factors other than the differentiation factors set out above.

SMAD2 and SMAD3 and/or SMAD2 and SMAD3 mediated intracellular signalling pathways may be stimulated by the first, second and third mesoderm induction media through the presence in the media of a first TGFIβ ligand. The first TGFIβ ligand may be Activin. Activin (Activin A: NCBI Gene ID: 3624 nucleic acid reference sequence NM_002192.2 GI: 62953137, amino acid reference sequence NP_002183.1 GI: 4504699) is a dimeric polypeptide which exerts a range of cellular effects via stimulation of the Activin/Nodal pathway (Vallier et al., Cell Science 118:4495-4509 (2005)). Activin is readily available from commercial sources (e.g. Stemgent Inc. MA USA; Miltenyi Biotec Gmbh, Del.). Conveniently, the concentration of Activin in a medium described herein may be from 1 to 100 ng/ml, preferably about 5 to 50 ng/ml.

The fibroblast growth factor (FGF) activity of the second and third mesoderm induction media may be provided by the presence of fibroblast growth factor (FGF) in the media. Fibroblast growth factor (FGF) is a protein factor which stimulates cellular growth, proliferation and cellular differentiation by binding to a fibroblast growth factor receptor (FGFR). Suitable fibroblast growth factors include any member of the FGF family, for example any one of FGF1 to FGF14 and FGF15 to FGF23. Preferably, the FGF is FGF2 (also known as bFGF, NCBI GeneID: 2247, nucleic acid sequence NM_002006.3 GI: 41352694, amino acid sequence NP_001997.4 GI: 41352695); FGF7 (also known as keratinocyte growth factor (or KGF), NCBI GeneID: 2247, nucleic acid sequence NM_002006.3 GI: 41352694, amino acid sequence NP_001997.4 GI: 41352695); or FGF10 (NCBI GeneID: 2247, nucleic acid sequence NM_002006.3 GI: 41352694, amino acid sequence NP_001997.4 GI: 41352695). Most preferably, the fibroblast growth factor is FGF2.

Conveniently, the concentration of FGF, such as FGF2 in a medium described herein may be from 0.5 to 50 ng/ml, preferably about 5 ng/ml. Fibroblast growth factors, such as FGF2, FGF7 and FGF10, may be produced using routine recombinant techniques or obtained from commercial suppliers (e.g. R&D Systems, Minneapolis, Min.; Stemgent Inc, USA; Miltenyi Biotec Gmbh, Del.).

SMAD1, SMAD5 and SMAD9 and/or SMAD1, SMAD5 and SMAD9 mediated intracellular signalling pathways may be stimulated by the second and third mesoderm induction media through the presence in the media of a second TGFIβ ligand.

The second TGFβ ligand may be a Bone Morphogenic Protein (BMP). Bone Morphogenic Proteins (BMPs) bind to Bone Morphogenic Protein Receptors (BMPRs) and stimulate intracellular signalling through pathways mediated by SMAD1, SMAD5 and SMAD9. Suitable Bone Morphogenic Proteins include any member of the BMP family, for example BMP2, BMP3, BMP4, BMP5, BMP6 or BMP7. Preferably the second TGFβ ligand is BMP2 (NCBI GeneID: 650, nucleic acid sequence NM_001200.2 GI: 80861484; amino acid sequence NP_001191.1 GI: 4557369) or BMP4 (NCBI GeneID: 652, nucleic acid sequence NM_001202.3 GI: 157276592; amino acid sequence NP_001193.2 GI: 157276593). Suitable BMPs include BMP4. Conveniently, the concentration of a Bone Morphogenic Protein, such as BMP2 or BMP4 in a medium described herein may be from 1 to 500 ng/ml, preferably about 10 ng/ml. BMPs may be produced using routine recombinant techniques or obtained from commercial suppliers (e.g. R&D, Minn., USA, Stemgent Inc, USA; Miltenyi Biotec Gmbh, DE.).

The GSK3β inhibition activity of the third mesoderm induction medium may be provided by the presence of a GSK3β inhibitor in the medium. GSK3β inhibitors inhibit the activity of glycogen synthase kinase 3β (Gene ID 2932: EC2.7.11.26). Preferred inhibitors specifically inhibit the activity of glycogen synthase kinase 3β. Suitable inhibitors include CHIR99021 (6-((2-((4-(2,4-Dichlorophenyl)-5-(4-methyl-1H-imidazol-2-yl)pyrimidin-2-yl)amino)ethyl)amino)nicotinonitrile; Ring D. B. et al., Diabetes, 52:588-595 (2003)) alsterpaullone, kenpaullone, BIO(6-bromoindirubin-3′-oxime (Sato et al Nat Med. 2004 Jan.; 10(1):55-63), SB216763 (3-(2,4-dichlorophenyl)-4-(1-methyl-1H-indol-3-yl)-1H-pyrrole-2,5-dione), Lithium and SB415286 (3-[(3-chloro-4-hydroxyphenyl)amino]-4-(2-nitrophenyl)-1H-pyrrole-2,5-dione; Coghlan et al Chem Biol. 2000 Oct.; 7(10):793-803). In some preferred embodiments, the GSK3β inhibitor is CHIR99021.Suitable glycogen synthase kinase 3β inhibitors may be obtained from commercial suppliers (e.g. Stemgent Inc. MA USA; Cayman Chemical Co. MI USA; Selleckchem, Mass. USA). For example, the third mesoderm induction medium may contain 0.1 to 100 μM, for example any of about 0.1, 0.25, 0.5, 1, 2, 3, 4, 5, 6, 7, 8, 9, 10, 11, 12, 13, 14, 15, 20, 25, 30, 35, 40, 45, 50, 60, 70, 80, 90 or 95 μM of a GSK3β inhibitor, such as CHIR99021, preferably about 10 μM.

In preferred embodiments, the first, second and third mesoderm induction media are chemically defined media. For example, the first mesoderm induction medium may consist of a chemically defined nutrient medium supplemented with an effective amount of activin, preferably activin A, for example 50 ng/ml activin A; the second mesoderm induction medium may consist of a chemically defined nutrient medium supplemented with an effective amount of activin preferably activin A, for example 5 ng/ml activin A, BMP, preferably BMP4, for example 10 ng/ml BMP4; and FGF, preferably bFGF (FGF2), for example 5 ng/ml bFGF; and the third mesoderm induction medium may consist of a chemically defined nutrient medium supplemented with an effective amount of activin preferably activin A, for example 5 ng/ml activin A, BMP, preferably BMP4, for example 10 ng/ml BMP4; FGF, preferably bFGF (FGF2), for example 5 ng/ml bFGF; and GSK3 inhibitor, preferably CHIR-99021 , for example 10μM CHIR-99021.

A chemically defined medium (CDM) is a nutritive solution for culturing cells which contains only specified components, preferably components of known chemical structure. A CDM is devoid of undefined components or constituents which include undefined components, such as feeder cells, stromal cells, serum, and complex extracellular matrices, such as matrigel™. For example, a CDM does not contain stromal cells, such as OP9 cells, expressing Notch ligands, such as DLL1 or DLL4.

The CDM or chemically defined nutrient medium may comprise a chemically defined basal medium. Suitable chemically defined basal media include Iscove's Modified Dulbecco's Medium (IMDM), Ham's F12, Advanced Dulbecco's modified eagle medium (DMEM) (Price et al Focus (2003), 25 3-6), Williams E

(Williams, G.M. et al Exp. Cell Research, 89, 139-142 (1974)), RPMI-1640 (Moore, G.E. and Woods L. K., (1976) Tissue Culture Association Manual. 3, 503-508) and StemPro™-34 (ThermoFisher Scientific).

The basal medium may be supplemented by serum-free culture medium supplements and/or additional components in the medium. Suitable supplements and additional components are described above and may include L-glutamine or substitutes, such as GlutaMAX-1198 , ascorbic acid, monothiolglycerol (MTG), antibiotics such as penicillin and streptomycin, human serum albumin, for example recombinant human serum albumin, such as Cellastim™ (Merck/Sigma) and Recombumin™ (albumedix.com), insulin, transferrin and 2-mercaptoethanol. A basal medium may be supplemented with a serum substitute, such as Knockout Serum Replacement (KOSR; Invitrogen).

The iPSCs may be cultured in the first mesoderm induction medium for 1 to 12 hours, for example any of 1, 2, 3, 4, 5, 6, 7, 8, 9, 10, 11 or 12 hours, preferably about 4 hours; then cultured in the second mesoderm induction medium for 30 to 54 hours, for example any of 35, 36, 37, 38, 39, 40, 41, 42, 43, 44, 45, 46, 47, 48, 49 or 50 hours, preferably about 44 hours; and then cultured in the third mesoderm induction medium for 36 to 60 hours, for example any of 37, 38, 39, 40, 41, 42, 43, 44, 45, 46, 47, 48, 49, 50, 51, 52 or 53 hours, preferably about 48 hours to produce a population of mesodermal cells.

Mesoderm cells are partially differentiated progenitor cells that are committed to mesodermal lineages and are capable of differentiation under appropriate conditions into all cell types in the mesenchyme (fibroblast), muscle, bone, adipose, vascular and haematopoietic systems. Mesoderm cells may express one or more mesodermal markers. For example, the mesoderm cells may express any one, two, three, four, five, six or all seven of Brachyury, Goosecoid, MixI1, KDR, FoxA2, GATA6 and PDGFaR.

In a second stage, mesoderm cells may be differentiated into haemogenic endothelial (HE) cells by culturing the population of mesoderm cells under suitable conditions to promote HE differentiation. For example, the iPSC derived mesoderm cells may be cultured in an HE induction medium.

A suitable HE induction medium may (i) stimulate cKIT receptor (CD117; KIT receptor tyrosine kinase) and/or cKIT receptor (CD117; KIT receptor tyrosine kinase) mediated signalling pathways and (ii) stimulate VEGFR and/or VEGF mediated signalling pathways. For example, the HE induction medium may comprise SCF and/or VEGF.

Vascular endothelial growth factor (VEGF) is a protein factor of the PDGF family which binds to VEGFR tyrosine kinase receptors and stimulates vasculogenesis and angiogenesis. Suitable VEGFs include any member of the VEGF family, for example any one of VEGF-A to VEGF-D and PIGF. Preferably, the VEGF is VEGF-A (also known as VEGF, NCBI Gene ID: 7422, nucleic acid sequence NM_001025366.2, amino acid sequence NP_001020537.2). Preferably, the VEGFR mediated signalling pathways are VEGFR2 (KDR/Flk-1) mediated signalling pathways. VEGF is readily available from commercial sources (e.g. R&D Systems, USA). Conveniently, the concentration of VEGF in an HE induction medium described herein may be from 1 to 100 ng/ml, for example any of about 5, 7, 10, 12, 15, 17, 20, 25, 30, 35, 40, 45 or 50 ng/ml preferably about 15 ng/ml.

In some examples of HE induction media, VEGF may be replaced by a VEGF activator or agonist that stimulates VEGFR mediated signalling pathways. Suitable VEGF activators are known in the art and include proteins, such as gremlin (Mitola et al (2010) Blood 116(18) 3677-3680) nucleic acids, such as shRNA (e.g. Turunen et al Circ Res. 2009 Sep. 11; 105(6):604-9), CRISPR-based plasmids (e.g. VEGF CRISPR activation plasmid; Santa Cruz Biotech, USA), antibodies and small molecules.

Stem cell factor (SCF) is a cytokine that binds to the KIT receptor (KIT proto-oncogene, receptor tyrosine kinase) (CD117; SCFR) and is involved in haematopoiesis. SCF (also called KITLG, NCBI GeneID: 4254) may have the reference nucleic acid sequence NM_000899.5 or NM_03994.5 and the reference amino acid sequence NP_000890.1 or NP_003985.5. SCF is readily available from commercial sources (e.g. R&D Systems, USA). Conveniently, the concentration of SCF in an HE induction medium described herein may be from 1 to 1000 ng/ml, for example any of about 10, 20, 30, 40, 50, 60, 70, 80, 90, 100, 110, 120, 130, 140, 150, 200, 250, 300, 350, 400, 450, 500, 600, 700, 800, 900 ng/ml, preferably about 100 ng/ml.

In preferred embodiments, the HE induction medium is a chemically defined medium. For example, the HE induction medium may consist of a chemically defined nutrient medium supplemented with effective amounts of VEGF, for example 15 ng/ml VEGF; and SCF, for example 100 ng/ml SCF. Preferably, mesoderm cells are cultured in an HE induction medium consisting of a chemically defined nutrient medium and two differentiation factors, wherein the two differentiation factors being SCF and VEGF.

Suitable chemically defined nutrient media are described above and include StemPro™-34 PLUS (ThermoFisher Scientific) or a basal medium such as IMDM supplemented with albumin, insulin, selenium transferrin, and lipids as described below.

The mesoderm cells may be cultured in the HE induction medium for 2 to 6 days, for example any of 2, 3, 4, 5 or 6 days, preferably about 4 days, to produce a population of HECs.

Haemogenic endothelial cells (HECs) are partially differentiated endothelial progenitor cells that have hematopoietic potential and are capable of differentiation under appropriate conditions into haematopoietic lineages. HECs may express CD34 and may, in some embodiments, not express CD73 or CXCR4 (CD184). For example, in some embodiments, HECs may have the phenotype CD34+ CD73−, or CD34+ CD73− CXCR4−.

In a third stage, haemogenic endothelial cells (HECs) may be differentiated into haematopoietic progenitor cells (HPCs) by culturing the population of HECs under suitable conditions to promote haematopoietic differentiation. For example, the HECs may be cultured in a haematopoietic induction medium.

A suitable haematopoietic induction medium may stimulate the following (i) cKIT receptor (CD117; KIT receptor tyrosine kinase) and/or cKIT receptor (CD117; KIT receptor tyrosine kinase) mediated signalling pathways , (ii) VEGFR and/or VEGFR mediated signalling pathways, preferably VEGFR2 and/or VEGFR2 mediated signalling pathways, (iii) MPL (CD110) and/or MPL (CD110) mediated signalling pathways (iv)

FLT3 and/or FLT3 mediated signalling pathways (v) IGF1 R and/or IGF1R mediated signalling pathways (vi) SMAD1, 5 and 9 and/or SMAD1, 5 and 9 mediated signalling pathways (vii) Hedgehog and/or Hedgehog signalling pathways (viii) EpoR and/or EpoR mediated signalling pathway and (ix) AGTR2 and/or AGTR2 mediated signalling pathways. A suitable haematopoietic induction medium may also inhibit the AGTR1 (angiotensin II type 1 receptor (AT₁)) and/or AGTR1 (angiotensin II type 1 receptor (AT₁)) mediated signaling pathway. A suitable haematopoietic induction medium may also have interleukin (IL) activity and FGF activity.

For example, a haematopoietic induction medium may comprise the differentiation factors: VEGF, SCF, Thrombopoietin (TPO), FIt3 ligand (FIt3L), IL-3, IL-6, IL-7, IL-11, IGF-1, BMP, FGF, Sonic hedgehog (SHH), erythropoietin (EPO), angiotensin II, and an angiotensin II type 1 receptor (AT₁) antagonist. An example of a suitable haematopoietic induction medium is the Stage 3 medium shown in Table 1 below.

Thrombopoietin (TPO) is a glycoprotein hormone that regulates platelet production. TPO (also called THPO, NCBI Gene ID: 7066) may have the reference nucleic acid sequence NM_000460.4 and the reference amino acid sequence NP_000451.1. TPO is readily available from commercial sources (e.g.

R&D Systems, USA; Miltenyi Biotec Gmbh, DE). Conveniently, the concentration of TPO in a haematopoietic induction medium described herein may be from 3 to 300 ng/ml, for example any of about 3, 4, 5, 6, 7, 8, 9, 10, 11, 12, 13, 14, 15, 20, 25, 27, 30, 32, 35, 40, 45, 50, 60, 70, 80, 90 or 100, 110, 120, 130, 140, 150, 160, 170, 180, 190, 200, 225, 250, 275 or 300 ng/ml, preferably about 30 ng/mil.

FIt3 ligand (Fms-related tyrosine kinase 3 ligand or FLT3L) is a cytokine with haematopoietic activity which binds to the FLT3 receptor and stimulates the proliferation and differentiation of progenitor cells. FIt3 ligand (also called FLT3LG, NCBI GeneID: 2323) may have the reference nucleic acid sequence NM_001204502.2 and the reference amino acid sequence NP_001191431.1. FIt3 is readily available from commercial sources (e.g. R&D Systems, USA; Miltenyi Biotec Gmbh, DE). Conveniently, the concentration of FIt3 ligand in a haematopoietic induction medium described herein may be from 0.25 to 250ng/ml, for example any of about 0.1, 0.25, 0.5, 1, 2, 3, 4, 5, 6, 7, 8, 9, 10, 11, 12, 13, 14, 15, 20, 25, 30, 35, 40, 45, 50, 60, 70, 80, 90 or 100, 110, 120, 130, 140, 150, 160, 170, 180, 190, 200, 210, 220, 230 or 240 ng/ml, preferably about 25 ng/ml.

Interleukins (ILs) are cytokines that play major roles in immune development and function. ILs in a haematopoietic induction medium may include IL-3, IL-6, IL-7, and IL-11.

IL-3 (also called IL3 or MCGF, NCBI GeneID: 3562) may have the reference nucleic acid sequence NM_000588.4 and the reference amino acid sequence NP_000579.2. IL-3 is readily available from commercial sources (e.g. R&D Systems, USA; Miltenyi Biotec Gmbh, DE). Conveniently, the concentration of IL-3 in a haematopoietic induction medium described herein may be from 0.25 to 250 ng/ml, for example any of about 0.1, 0.25, 0.5, 1, 2, 3, 4, 5, 6, 7, 8, 9, 10, 11, 12, 13, 14, 15, 20, 25, 30, 35, 40, 45, 50, 60, 70, 80, 90 or 100, 110, 120, 130, 140, 150, 160, 170, 180, 190, 200, 210, 220, 230 or 240 ng/ml, preferably about 25 ng/ml. IL-6 (also called IL6 or HGF, NCBI GeneID: 3569) may have the reference nucleic acid sequence NM_000600.5 and the reference amino acid sequence NP_000591.5. IL-6 is readily available from commercial sources (e.g. R&D Systems, USA; Miltenyi Biotec Gmbh, DE). Conveniently, the concentration of IL-6 in a haematopoietic induction medium described herein may be from 0.1 to 10 ng/ml, for example any of about 0.1, 0.25, 0.5, 1, 2, 3, 4, 5, 6, 7, 8, 9, 10, 11, 12, 13, 14, 15, 20, 25, 30, 35, 40, 45, 50, 60, 70, 80, 90 or 95 ng/ml, preferably about 10 ng/ml.

IL-7 (also called IL7, NCBI GeneID: 3574) may have the reference nucleic acid sequence NM_000880.4 and the reference amino acid sequence NP_000871.1. IL-7 is readily available from commercial sources (e.g. R&D Systems, USA; Miltenyi Biotec Gmbh, DE). Conveniently, the concentration of IL-7 in a haematopoietic induction medium described herein may be from 0.1 to 100 ng/ml, for example any of about 0.1, 0.25, 0.5, 0.75, 1, 2, 3, 4, 5, 6, 7, 8, 9, 10, 11, 12, 13, 14, 15, 20, 25, 30, 35, 40, 45, 50, 60, 70, 80, 90 or 95 ng/ml, preferably about 10 ng/ml.

IL-11 (also called AGIF, NCBI GeneID: 3589) may have the reference nucleic acid sequence NM_000641.4 and the reference amino acid sequence NP_000632.1. IL-11 is readily available from commercial sources (e.g. R&D Systems, USA; Miltenyi Biotec Gmbh, DE). Conveniently, the concentration of IL-11 ligand in a haematopoietic induction medium described herein may be from 0.5 to 100 ng/ml, for example any of about 0.1, 0.25, 0.5, 0.75, 1, 2, 3, 4, 5, 6, 7, 8, 9, 10, 11, 12, 13, 14, 15, 20, 25, 30, 35, 40, 45, 50, 60, 70, 80, 90 or 95 ng/ml, preferably about 5 ng/ml.

Insulin-like growth factor 1 (IGF-1) is a hormone that binds to the tyrosine kinases IGF-1 receptor (IGF1R) and insulin receptor and activates the multiple signalling pathways. IGF-1(also called IGF or MGF, NCBI GeneID: 3479) may have the reference nucleic acid sequence NM_000618.5 and the reference amino acid sequence NP_000609.1. IGF-1 is readily available from commercial sources (e.g. R&D Systems,

USA). Conveniently, the concentration of IGF-1 in a haematopoietic induction medium described herein may be from 0.25 to 250 ng/ml, for example any of about 0.1, 0.25, 0.5, 1, 2, 3, 4, 5, 6, 7, 8, 9, 10, 11, 12, 13, 14, 15, 20, 25, 30, 35, 40, 45, 50, 60, 70, 80, 90 or 100, 110, 120, 130, 140, 150, 160, 170, 180, 190, 200, 210, 220, 230 or 240 ng/ml, preferably about 25 ng/ml.

Sonic hedgehog (SHH) is a ligand of the hedgehog signalling pathway that regulates vertebrate organogenesis. SHH (also called TPT or HHG1, NCBI GeneID: 6469) may have the reference nucleic acid sequence NM_000193.4 and the reference amino acid sequence NP_000184.1. SHH is readily available from commercial sources (e.g. R&D Systems, USA; Miltenyi Biotec Gmbh, DE). Conveniently, the concentration of SHH in a haematopoietic induction medium described herein may be from 0.25 to 250 ng/ml, for example any of about 0.1, 0.25, 0.5, 1, 2, 3, 4, 5, 6, 7, 8, 9, 10, 11, 12, 13, 14, 15, 20, 25, 30, 35, 40, 45, 50, 60, 70, 80, 90 or 100, 110, 120, 130, 140, 150, 160, 170, 180, 190, 200, 210, 220, 230 or 240 ng/ml, preferably about 25 ng/ml.

Erythropoietin (EPO) is a glycoprotein cytokine that binds to the erythropoietin receptor (EpoR) and stimulates erythropoiesis. EPO (also called DBAL, NCBI GeneID: 2056) may have the reference nucleic acid sequence NM_000799.4 and the reference amino acid sequence NP_000790.2. EPO is readily available from commercial sources (e.g. R&D Systems, USA; PreproTech, USA). Conveniently, the concentration of EPO in haematopoietic induction medium described herein may be from 0.02 to 20 U/ml, for example any of about 0.01, 0.025, 0.05, 0.075, 0.1, 0.5, 0.75, 1.0, 1.5, 2.0, 2.5, 3, 4, 5, 6, 7, 8, 9, 10 13, 15, 17, or 19 U/ml, preferably about 2 U/ml.

Angiotensin II is a heptapeptide hormone that is formed by the action of angiotensin converting enzyme (ACE) on angiotensin I. Angiotensin II stimulates vasoconstriction. Angiotensin I and II are formed by the cleavage of angiotensinogen (also called AGT, NCBI GeneID: 183), which may have the reference nucleic acid sequence NM_000029.4 and the reference amino acid sequence NP_000020.1. Angiotensin 11 is readily available from commercial sources (e.g. R&D Systems, USA; Tocris, USA). Conveniently, the concentration of angiotensin II in a haematopoietic induction medium described herein may be from 0.05 to 50 ng/ml, for example any of about 0.01, 0.025, 0.05, 0.075, 0.1, 0.5, 0.75, 1.0, 1.5, 2.0, 2.5, 3, 4, 5, 6, 7, 8, 9, 10, 15, 20, 30, 40 or 50 ng/ml preferably about 5 ng/ml.

Angiotensin II type 1 receptor (AT₁) antagonists (ARBs) are compounds that selectively block the activation of AT₁ receptor (AGTR1; Gene ID 185). Suitable AT₁ antagonists include losartan (2-Butyl-4-chloro-1-{[2′-(1H-tetrazol-5-yl)-4-biphenylyl]methyl}-1H-imidazol-5-yl)methanol), valsartan ((2S)-3-Methyl-2-(pentanoyl{[2′-(1H-tetrazol-5-yl)biphenyl-4-yl]methyl}amino)butanoic acid), and telmisartan (4′[(1,4′-Dimethyl-2′-propyl[2,6′-bi-1H-benzimidazol]-1′-yl)methyl][1,1′-biphenyl]-2-carboxylic acid. In some preferred embodiments, the AT₁ antagonist is losartan. Suitable AT₁ antagonists may be obtained from commercial suppliers (e.g. Tocris, USA; Cayman Chemical Co. MI USA). Conveniently, the concentration of angiotensin II type 1 receptor (AT₁) antagonist in a haematopoietic induction medium described herein may be from 1 to 1000 μM, for example any of about 10, 20, 30, 40, 50, 60, 70, 80, 90, 100, 110, 120, 130, 140, 150, 160, 170, 180, 190, 200, 250, 300, 350, 400, 450, 500, 600, 700, 800, 900 μM, preferably about 100 μM.

In preferred embodiments, the haematopoietic induction medium is a chemically defined medium. For example, the haematopoietic induction medium may consist of a chemically defined nutrient medium supplemented with effective amounts of VEGF, for example 15ng/m1; SCF, for example 100 ng/ml; thrombopoietin (TPO), for example 30 ng/ml; FIt3 ligand (FLT3L), for example 25 ng./ml; IL-3, for example 25 ng/ml; IL-6, for example 10 ng/ml; IL-7, for example 10 ng/ml; IL-11, for example 5 ng/ml; IGF-1, for example 25 ng/ml; BMP, for example BMP4 at 10 ng/ml; FGF, for example bFGF at 5 ng/ml; Sonic hedgehog (SHH), for example 25 ng/ml; erythropoietin (EPO), for example 2 u/ml; angiotensin II, for example 10 μg/ml, and an angiotensin II type 1 receptor (AT₁) antagonist, for example losartan, at 100 μM.

Suitable chemically defined nutrient media are described above and include StemPro™-34 PLUS (ThermoFisher Scientific) or a basal medium such as IMDM supplemented with albumin, insulin, selenium transferrin, and lipids as described below.

The HECs may be cultured in the haematopoietic induction medium for 8-21 days, for example any of about 9, 10, 11, 12, 13, 14, 15, 16, 17, 18, 19 or 20 days, preferably about 16 days, to produce the population of HPCs.

HPCs (also called hematopoietic stem and progenitor cells or HSPCs) are multipotent stem cells that are committed to a hematopoietic lineage and are capable of further hematopoietic differentiation into all blood cell types including myeloid and lymphoid lineages, including monocytes, B cells, NK cells, NKT cells, TILs, and T cells. HPCs may express CD34. HPCs may co-express CD45. HPCs may also co-express CD117, CD133, CD45 and FLK1 (also known as KDR or VEGFR2). HPCs may be negative for expression of CD38 and other lineage specific markers. For example, HPCs may display one or more, preferably all of CD34+ CD133+ CD45+ FLK1+ CD38−.

HECs and HPCs may be generated from iPSCs without any intermediate purification or isolation of individual cells or subsets of cells from the population of differentiating cells. For example, cells of a particular class, type, lineage or phenotype may not be separated from other cells in the population of differentiating cells. Conversely, cells in the population of differentiating cells that are not in a particular class, type, lineage or phenotype may not be separated from cells in the population of the particular class, type, lineage or phenotype. Purification or isolation steps may increase the proportion of cells in a population that display a particular class, type, lineage or phenotype. Such steps are unnecessary in the methods described herein in order to produce haemogenic progenitor cells. Techniques for cell purification or isolation include cell sorting or gating techniques, such as magnetic activated cell sorting (MACS), fluorescence activated cell sorting (FACS) and microfluidic cell sorting, and are well established in the art.

The population of iPSCs may be differentiated into HECs and HPCs as described herein in a single culture vessel using the different culture media described herein to drive differentiation. Target cells capable of differentiating into HECs or HPCs may not be separated from non-target cells in the population during the differentiation. For example, target cells or non-target cells may not be isolated from the cell population in order to increase the concentration or proportion of target cells.

Following the generation of HPCs from HECs, HPCs expressing one or more cell surface markers, such as CD34, may be purified or isolated from other cells in the population, for example by magnetic activated cell sorting (MACS) or other cell purification or isolation technique, before being subjected to further differentiation. For example, a population of CD34+ HPCs may be purified. The CD34+ HPCs may be purified after 8 days, for example 8-10 days, culture in the HE induction medium. The CD34+ HPCs may be purified after 16 days of differentiation, for example on day 16 to day 18, i.e. day 16, 17 or 18, of the differentiation method.

Preferably, HPCs produced as described herein are used to generate populations of T cells. A population of T cells may be produced by a method comprising;
differentiating a population of iPSCs into HPCs as described above, and
differentiating the HPCs into T cell progenitors.

Haematopoietic progenitor cells (HPCs) may be differentiated into progenitor T cells by culturing the population of HPCs under suitable conditions to promote lymphoid differentiation. For example, the haematopoietic progenitor cells may be cultured in a lymphoid expansion medium.

Haematopoietic progenitor cells (HPCs) may be differentiated into progenitor T cells and DP (double positive) T cells in the absence of stromal cells, such as OP9-D14 stromal cells, feeder cells or serum.

A lymphoid expansion medium is a cell culture medium that promotes the lymphoid differentiation of HPCs into progenitor T cells.

A suitable lymphoid expansion medium may (i) stimulate cKIT receptor (CD117; KIT receptor tyrosine kinase) and/or cKIT receptor (CD117; KIT receptor tyrosine kinase) mediated signalling pathways, (ii) stimulate MPL (CD110) and/or MPL (CD110) mediated signalling pathways (iii) FLT3 and/or FLT3 mediated signalling pathways and (iv) have interleukin (IL) activity. For example, a lymphoid expansion medium may comprise the differentiation factors SCF, FLT3L, TPO and IL7.

In preferred embodiments, the lymphoid expansion medium is a chemically defined medium. For example, the lymphoid expansion medium may consist of a chemically defined nutrient medium supplemented with effective amounts of the above differentiation factors. Suitable lymphoid expansion media are well-known in the art and include Stemspan™ SFEM II (Cat #9605; StemCell Technologies Inc, CA).with Stemspan™ lymphoid expansion supplement (Cat #9915; StemCell Technologies Inc, CA).

The HPCs may be cultured on a surface during differentiation into progenitor T cells. For example, the HPCs may be cultured on a surface of a culture vessel, bead or other biomaterial or polymer.

Preferably, the surface may be coated with a factor that stimulates Notch signalling, for example a Notch ligand, such as Delta-like 1 (DLL1) or Delta-like 4 (DLL4). Suitable Notch ligands are well-known in the art and available from commercial suppliers.

The surface may also be coated with an extracellular matrix protein, such as fibronectin, vitronectin, laminin or collagen and/or one or more cell surface adhesion proteins, such as VCAM1.

In some embodiments, the surface for HPC culture may have a coating that comprises a factor that stimulates Notch signalling, for example a Notch ligand, such as DLL4, an extracellular matrix protein, such as vitronectin, and a cell surface adhesion protein, such as VCAM1. In some embodiments, the surface for HPC culture may have a coating that comprises a factor that stimulates Notch signalling, for example a Notch ligand, such as DLL4, without the extracellular matrix protein or cell surface adhesion protein.

The surface may be coated with an extracellular matrix protein, factor that stimulates Notch signalling and/or a cell surface adhesion protein by contacting the surface with a coating solution. For example, the coating solution may be incubated on the surface under suitable conditions to coat the surface. Conditions may, for example, include about 2 hours at room temperature. Coating solutions comprising an extracellular matrix protein and a factor that stimulates Notch signalling are available from commercial suppliers (StemSpan™ Lymphoid Differentiation Coating Material; Cat #9925; Stem Cell Technologies Inc, CA).

The HPCs may be cultured in the lymphoid expansion medium on the substrate for a time sufficient for the HPCs to differentiate into progenitor T cells. For example, the HPCs may be cultured for 2-6 weeks or 2-4 weeks, 2-5 weeks, preferably 3 weeks.

Progenitor T cells are multi-potent lymphopoietic progenitor cells that are capable of giving rise to aβ T cells, yδ T cells, tissue resident T cells and NK T cells. Progenitor T cells may commit to the aβ T cell lineage after pre-TCR selection in the thymus. Progenitor T cells may be capable of in vivo thymus colonization and may be capable of committing to the αβ T cell lineage after pre-TCR selection in the thymus. Progenitor T cells may also be capable of maturation into cytokine-producing CD3+ T-cells.

Progenitor T cells may express CD5 and CD7 i.e. the progenitor T cells may have a CD5+CD730 phenotype. Progenitor T cells may also co-express CD44, CD25 and CD2. For example, progenitor T cells may have a CD5+, CD7+CD44+, CD25+CD2+phenotype. Progenitor T cells may also co-express CD45. Progenitor T cells may lack expression, for example cell surface expression, of CD3, CD4 and CD8.

In a fifth stage, progenitor T cells may be matured into T cells by culturing the population of progenitor T cells under suitable conditions to promote T cell maturation. For example, the progenitor T cells may be cultured in a T cell maturation medium.

A T cell maturation medium is a cell culture medium that promotes the maturation of progenitor T cells into mature T cells. A suitable T cell maturation medium may (i) stimulate cKIT receptor (CD117; KIT receptor tyrosine kinase) and/or stimulate cKIT receptor (CD117; KIT receptor tyrosine kinase) mediated signalling pathways (ii) stimulate FLT3 and/or FLT3 mediated signalling pathways and (iii) have interleukin (IL) activity. For example, a T cell maturation medium may comprise the differentiation factors SCF, FLT3L, and IL7.

In preferred embodiments, the T cell maturation medium is a chemically defined medium. For example, the T cell maturation medium may consist of a chemically defined nutrient medium supplemented with effective amounts of the above differentiation factors. Suitable T cell maturation media are well-known in the art and include Stemspan™ SFEM II (Cat #9605; StemCell Technologies Inc, CA) with Stemspan™ T cell maturation supplement (Cat #9930; StemCell Technologies Inc, CA) and other media suitable for expansion of PBMCs and CD3+ cells, such as ExCellerate Human T cell expansion medium (R&D Systems, USA). Other suitable T cell maturation media may include a basal medium such as IMDM, supplemented with ITS, albumin and lipids, as described elsewhere herein and further supplemented with effective amounts of the above differentiation factors.

The progenitor T cells may be cultured on a surface. For example, the progenitor T cells may be cultured on a surface of a culture vessel, bead or other biomaterial or polymer.

Preferably, the surface may be coated with a factor that stimulates Notch signalling, for example a Notch ligand, such as Delta-like 1 (DLL1) or Delta-like 4 (DLL4). Suitable Notch ligands are well-known in the art and available from commercial suppliers. The surface may also be coated with an extracellular matrix protein, such as fibronectin, vitronectin, laminin or collagen and/or one or more cell surface adhesion proteins, such as VCAM1. Suitable coatings are well-known in the art and described elsewhere herein.

The progenitor T cells may be cultured in the T cell maturation medium on the substrate for a time sufficient for the progenitor T cells to mature into T cells. For example, the progenitor T cells may be cultured for 1-4 weeks, preferably 2 or 3 weeks.

T cells (also called T lymphocytes) are white blood cells that play a central role in cell-mediated immunity. T cells can be distinguished from other lymphocytes by the presence of a T cell receptor (TCR) on the cell surface.

There are several types of T cells, each type having a distinct function.

T helper cells (TH cells) are known as CD4+ T cells because they express the CD4 surface glycoprotein. CD4+ T cells play an important role in the adaptive immune system and help the activity of other immune cells by releasing T cell cytokines and helping to suppress or regulate immune responses. They are essential for the activation and growth of cytotoxic T cells.

Cytotoxic T cells (TC cells, CTLs, killer T cells, cytolytic T cells) are known as CD8+ T cells because they express the CD8 surface glycoprotein. CD8+ T cells act to destroy virus-infected cells and tumour cells. Most CD8+ T cells express TCRs that can recognise a specific antigen displayed on the surface of infected or damaged cells by a class I MHC molecule, or may recognise specific antigen or peptide thereof independent of presentation by MHC, such T cells may be produced according to the methods of the present invention. Specific binding of the TCR and CD8 glycoprotein to the antigen and MHC molecule leads to T cell-mediated destruction of the infected or damaged cells.

T cells produced as described herein may be mature CD3+ T cells. In some embodiments, T cells may also express CD45 and CD28.

T cells produced as described herein may be yδ T cells, αβ T cells or NKT cells. In some preferred embodiments, the T cells produced as described herein are αβ T cells. Following maturation of progenitor T cells (stage 5), the population of T cells may be predominantly double positive (DP) CD4+CD8+ T cells.

In a sixth stage, the population of T cells may be activated and/or expanded to produce or increase the proportion of single positive CD4+ T cells, or more preferably single positive CD8+ T cells. Suitable methods for activating and expanding T cells are well-known in the art. For example, T cells may be exposed to a T cell receptor (TCR) agonist under appropriate culture conditions. Suitable TCR agonists include ligands, such as peptides displayed on a class I or II MHC molecule (MHC-peptide complexes) on the surface of a bead or an antigen presenting cell, such as a dendritic cell, and soluble factors, such as anti-TCR antibodies for example antibody CD28 antibodies, and multimeric MHC-peptide complexes, such as MHC-peptide tetramers, pentamers or dextramers.

Activation refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division.

An anti-TCR antibody may specifically bind to a component of the TCR, such as εCD3, αCD3 or αCD28. Anti-TCR antibodies suitable for TCR stimulation are well-known in the art (e.g. OKT3) and available from commercial suppliers (e.g. eBioscience CO USA). In some embodiments, T cells may be activated by exposure to anti-αCD3 antibodies and IL2, IL-7, or IL15. More preferably, T cells are activated by exposure to anti-αCD3 antibodies and anti-αCD28 antibodies. The activation may occur in the presence or absence of CD14+ monocytes. The T cells may be activated with anti-CD3 and anti-CD28 antibody coated beads. For example, PBMCs or T cell subsets including CD4+ and/or CD8+ cells may be activated, without feeder cells (antigen presenting cells) or antigen, using antibody coated beads, for examplmagnetic beads coated with anti-CD3 and anti-CD28 antibodies, such as Dynabeads® Human T-Activator CD3/CD28 (ThermoFisher Scientific). In other embodiments, soluble tetrameric antibody complexes that bind CD3, CD28 and CD2 cell surface ligands, such as ImmunoCult™ Human CD3/CD28/CD2 T Cell Activator or Human CD3/CD28 T Cell Activator, may be used to activate the T cells. In other embodiments, T cells may be activated with an MHC-peptide complex, preferably a multimeric MHC-peptide complex, optionally in combination with an anti-CD28 antibody.

T cells expressing a chimeric antigen receptor may be activated using a soluble antigen to the receptor. The antigen may be in a multimeric form or on the surface of a bead and may optionally be used in conjunction with an anti-TCR antibody, such as an anti-CD28 antibody.

In some embodiments, double positive CD4+CD8+ T cells, may be cultured in a T cell maturation medium as described herein supplemented with IL-15. The medium may be further supplemented with a T cell receptor (TCR) agonist, for example one or more anti-TCR antibodies, such as anti-αCD3 antibodies, and anti-αCD28 antibodies, as described above.

The double positive CD4+ CD8+ T cells may be cultured using any convenient technique to produce the expanded population. Suitable culture systems include stirred tank fermenters, airlift fermenters, roller bottles, culture bags or dishes, and other bioreactors, in particular hollow fibre bioreactors. The use of such systems is well-known in the art

T cells, produced as described herein may express an antigen receptor that binds a target antigen. For example, the antigen receptor may bind specifically to cancer cells that express a tumor antigen. The T cells may be useful for example in immunotherapy, as described below.

The antigen receptor may be a T cell receptor (TCR). TCRs are disulphide-linked membrane anchored heterodimeric proteins, typically comprising highly variable alpha (α) and beta (β) chains expressed as a complex with invariant CD3 chain molecules. T cells expressing these type of TCRs are referred to as α:β (or αβ) T cells. A minority of T cells express an alternative TCR comprising variable gamma (γ) and delta (δ) chains and are referred to as γδ T cells.

Suitable TCRs bind specifically to a major histocompatibility complex (MHC) on the surface of cancer cells that displays a peptide fragment of a tumour antigen. An MHC is a set of cell-surface proteins which allow the acquired immune system to recognise ‘foreign’ molecules. Proteins are intracellularly degraded and presented on the surface of cells by the MHC. MHCs displaying ‘foreign’ peptides, such a viral or cancer associated peptides, are recognised by T cells with the appropriate TCRs, prompting cell destruction pathways. MHCs on the surface of cancer cells may display peptide fragments of tumour antigen i.e. an antigen which is present on a cancer cell but not the corresponding non-cancerous cell. T cells which recognise these peptide fragments may exert a cytotoxic effect on the cancer cell.

In some embodiments, the TCR expressed by the T cells may be naturally expressed (i.e. an endogenous TCR). For example, the T cells may be produced as described herein from iPSCs that are derived from Tumour Infiltrating Lymphocytes (TILs). TILs, for example tumour resident CD3+ CD8+ cells, may be obtained from an individual with a cancer condition using standard techniques. Alternatively, the T cells may be produced as described herein from iPSCs that are derived from T cells that bind to a peptide fragment of the target antigen displayed on a class I or II MHC molecule on the surface of an antigen presenting cell, such as a dendritic cell; or a population of T cells produced as described herein may be screened for binding to a peptide fragment of the target antigen displayed on a class I or II MHC molecule, and T cells that bind to the displayed peptide fragment identified.

In other embodiments, the TCR is not naturally expressed by the cells (i.e. the TCR is exogenous or heterologous). Suitable heterologous TCR may bind specifically to class I or II MHC molecules displaying peptide fragments of a target antigen. For example, the T cells may be modified to express a heterologous αβ TCR that binds specifically to class I or II MHC molecules displaying peptide fragments of a tumour antigen expressed by the cancer cells in a cancer patient. Tumour antigens expressed by cancer cells in the cancer patient may identified using standard techniques. Preferred tumour antigens include NY-ESO1, PRAME, alpha-fetoprotein (AFP), MAGE A4, MAGE A1, MAGE A10 and MAGE B2, most preferably NY-ESO-1, MAGE-A4 and MAGE-A10.

Suitable TCRs may include unconventional TCRs, for example non-MHC dependent TCRs that bind recognize non-peptide antigens displayed by monomorphic antigen-presenting molecules, such as CD1 and MR1; NKT cell TCRs and intraepithelial lymphocyte (IEL) TCRs. In some embodiments, the TCR may recognise target antigen or peptide fragment of target antigen on the cancer cell independently of MHC presentation.

A heterologous TCR may be a synthetic or artificial TCR i.e. a TCR that does not exist in nature. For example, a heterologous TCR may be engineered to increase its affinity or avidity for a tumour antigen (i.e. an affinity enhanced TCR). The affinity enhanced TCR may comprise one or more mutations relative to a naturally occurring TCR, for example, one or more mutations in the hypervariable complementarity determining regions (CDRs) of the variable regions of the TCR α and β chains. These mutations increase the affinity of the TCR for MHCs that display a peptide fragment of a tumour antigen expressed by cancer cells. Suitable methods of generated affinity enhanced TCRs include screening libraries of TCR mutants using phage or yeast display and are well known in the art (see for example Robbins et al J Immunol (2008) 180(9):6116; San Miguel et al (2015) Cancer Cell 28 (3) 281-283; Schmitt et al (2013) Blood 122 348-256; Jiang et al (2015) Cancer Discovery 5 901). Preferred affinity enhanced TCRs may bind to cancer cells expressing one or more of the tumour antigens NY-ESO1, PRAME, alpha-fetoprotein (AFP), MAGE A4, MAGE A1, MAGE A10 and MAGE B2.

Alternatively, the antigen receptor may be a chimeric antigen receptor (CAR). CARs are artificial receptors that are engineered to contain an immunoglobulin antigen binding domain, such as a single-chain variable fragment (scFv). A CAR may, for example, comprise an scFv fused to a TCR CD3 transmembrane region and endodomain. An scFv is a fusion protein of the variable regions of the heavy (V_{H}) and light (V_{L}) chains of immunoglobulins, which may be connected with a short linker peptide of approximately 10 to 25 amino acids (Huston J.S. et al. Proc Natl Acad Sci USA 1988; 85(16):5879-5883). The linker may be glycine-rich for flexibility, and serine or threonine rich for solubility, and may connect the N-terminus of the V_{H} to the C-terminus of the V_{L}, or vice versa. The scFv may be preceded by a signal peptide to direct the protein to the endoplasmic reticulum, and subsequently the T cell surface. In the CAR, the scFv may be fused to a TCR transmembrane and endodomain. A flexible spacer may be included between the scFv and the TCR transmembrane domain to allow for variable orientation and antigen binding. The endodomain is the functional signal-transmitting domain of the receptor. An endodomain of a CAR may comprise, for example, intracellular signalling domains from the CD3 ζ-chain or from receptors such as CD28, 41 BB, or ICOS. A CAR may comprise multiple signalling domains, for example, but not limited to, CD3z-CD28-41 BB or CD3z-CD28-OX40.

The CAR may bind specifically to a tumour-specific antigen expressed by cancer cells. For example, the T cells may be modified to express a CAR that binds specifically to a tumour antigen that is expressed by the cancer cells in a specific cancer patient. Tumour antigens expressed by cancer cells in the cancer patient may identified using standard techniques.

Alternatively, the antigen receptor may be an NK cell receptor (NKCR).

Expression of a heterologous antigen receptor, such as a heterologous TCR, NKCR or CAR, may alter the immunogenic specificity of the T cells produced as described herein so that they recognise or display improved recognition for one or more target antigens, e.g. tumour antigens that are present on the surface of the cancer cells of an individual with cancer. In some embodiments, the T cells produced as described herein may display reduced binding or no binding to cancer cells in the absence of the heterologous antigen receptor. For example, expression of the heterologous antigen receptor may increase the affinity and/or specificity of the cancer cell binding of a T cell relative to T cells that do not express the antigen receptor.

The term “heterologous” refers to a polypeptide or nucleic acid that is foreign to a particular biological system, such as a host cell, and is not naturally present in that system. A heterologous polypeptide or nucleic acid may be introduced to a biological system by artificial means, for example using recombinant techniques. For example, heterologous nucleic acid encoding a polypeptide may be inserted into a suitable expression construct which is in turn used to transform a host cell to produce the polypeptide. A heterologous polypeptide or nucleic acid may be synthetic or artificial or may exist in a different biological system, such as a different species or cell type. An endogenous polypeptide or nucleic acid is native to a particular biological system, such as a host cell, and is naturally present in that system. A recombinant polypeptide is expressed from heterologous nucleic acid that has been introduced into a cell by artificial means, for example using recombinant techniques. A recombinant polypeptide may be identical to a polypeptide that is naturally present in the cell or may be different from the polypeptides that are naturally present in that cell.

T cells may be modified to express the heterologous antigen receptor, such as a TCR or CAR, by the introduction of heterologous encoding nucleic acid into cells at any stage in the method described herein. For example, heterologous encoding nucleic acid may be introduced into iPSCs, HPCs, mesoderm cells, HECs, or progenitor T cells. In some preferred embodiments, cells may be transduced with heterologous nucleic acid encoding an antigen receptor in lymphoid expansion medium, for example after 2 weeks culture in lymphoid expansion medium (stage 4) as described herein. Heterologous nucleic acid encoding an antigen receptor may encode all the sub-units of the receptor. For example, nucleic acid encoding a TCR may comprise a nucleotide sequence encoding a TCR a chain and a nucleotide sequence encoding a TCR β chain, or a nucleotide sequence encoding a TCR δ chain and a nucleotide sequence encoding a TCR γ chain.

Nucleic acid may be introduced into the cells by any convenient technique. When introducing or incorporating a heterologous nucleic acid into an iPSC, HPC or progenitor T cell, certain considerations must be taken into account, well-known to those skilled in the art. The nucleic acid to be inserted should be assembled within a construct or vector which contains effective regulatory elements which will drive transcription in the T cell. Many known techniques and protocols for manipulation and transformation of nucleic acid, for example in preparation of nucleic acid constructs, introduction of DNA into cells and gene expression are described in detail in Protocols in Molecular Biology, Second Edition, Ausubel et al. eds. John Wiley & Sons, 1992. In some embodiments, nucleic acid may be introduced into the cells by gene editing. For example, a DNA double strand break (DSB) at a target site may be induced by a CRISPR/Cas9 system and the repair of the DSB may introduce the heterologous nucleic acid into the cell genome at the target site or the nucleic acid may be introducing using an rAAV vector (AAV mediated gene editing; Hirsch et al 2014 Methods Mol Biol 1114 291-307).

Suitable techniques for introducing the expression vector into the iPSCs, HPCs or progenitor T cells are well known in the art and include calcium phosphate transfection, DEAE-Dextran, electroporation, liposome-mediated transfection, gene editing and transduction using retrovirus or other virus, e.g. vaccinia or lentivirus. Preferably, nucleic acid encoding the heterologous antigen receptor may be contained in a viral vector, most preferably a gamma retroviral vector or a lentiviral vector, such as a

VSVg-pseudotyped lentiviral vector. A method described herein may comprise transducing a population of cells, for example iPSCs, HPCs or progenitor T cells, with a viral vector to produce a transduced population of genetically modified cells. The cells may be transduced by contact with a viral particle comprising the nucleic acid. Viral particles for transduction may be produced according to known methods. For example, HEK293T cells may be transfected with plasmids encoding viral packaging and envelope elements as well as a lentiviral vector comprising the coding nucleic acid. A VSVg-pseudotyped viral vector may be produced in combination with the viral envelope glycoprotein G of the Vesicular stomatitis virus (VSVg) to produce a pseudotyped virus particle. For example, solid-phase transduction may be performed without selection by culture on retronectin-coated, retroviral vector-preloaded tissue culture plates.

Following production, the population of T cells, for example DP CD4+ CD8+ cells, SP CD4+ cells or SP CD8+ cells, may be isolated and/or purified. Any convenient technique may be used, including fluorescence-activated cell sorting (FACS) or magnetic-activated cell sorting using antibody coated magnetic particles (MACS).

The population of T cells, for example DP CD4+CD8+ cells, SP CD4+ cells or SP CD8+ cells, may be expanded and/or concentrated. Optionally, the population of T cells produced as described herein may be stored, for example by cryopreservation, before use.

A population of T cells may be admixed with other reagents, such as buffers, carriers, diluents, preservatives and/or pharmaceutically acceptable excipients. Suitable reagents are described in more detail below. A method described herein may comprise admixing the population of T cells with a pharmaceutically acceptable excipient. Pharmaceutical compositions suitable for administration (e.g. by infusion), include aqueous and non-aqueous isotonic, pyrogen-free, sterile injection solutions which may contain anti-oxidants, buffers, preservatives, stabilisers, bacteriostats, and solutes which render the formulation isotonic with the blood of the intended recipient; and aqueous and non-aqueous sterile suspensions which may include suspending agents and thickening agents. Examples of suitable isotonic vehicles for use in such formulations include Sodium Chloride Injection, Ringer's Solution, or Lactated Ringer's Injection. Suitable vehicles can be found in standard pharmaceutical texts, for example, Remington's Pharmaceutical Sciences, 18th edition, Mack Publishing Company, Easton, Pa., 1990.

In some preferred embodiments, the T cells, which may be DP CD4+CD8+ T cells, SP CD4+ T cells or preferably SP CD8+ T cells, may be formulated into a pharmaceutical composition suitable for intravenous infusion into an individual.

The term “pharmaceutically acceptable” as used herein pertains to compounds, materials, compositions, and/or dosage forms which are, within the scope of sound medical judgement, suitable for use in contact with the tissues of a subject (e.g., human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

An aspect of the invention provides a population of T cells, which may be for example DP CD4+CD8+ T cells, SP CD4+ T cells or SP CD8+ T cells, produced by a method described above.

The population of T cells may be for use as a medicament. For example, a population of mature T cells as described herein may be used in cancer immunotherapy therapy, for example adoptive T cell therapy.

Adoptive cellular therapy or adoptive immunotherapy refers to the adoptive transfer of human T lymphocytes that express antigen receptors that are specific for target cells. For example, human T lymphocytes may express TCRs that are specific for antigens expressed on target cells and/or TCRs that are specific for peptide MHC complexes expressed on target cells or chimeric antigen receptors (CAR) that are specific for antigens expressed on target cells.

This can be used to treat a range of diseases depending upon the target chosen, e.g., tumour specific antigens to treat cancer. Adoptive cellular therapy involves removing a portion of a donor's or the patient's cells, for example, white blood cells. The cells are then used to generate iPSCs in vitro and these iPSCs are used to efficiently generate T cells that are specific for antigens expressed on target cells and/or specific for peptide MHC complexes on target cells as described herein. The T cells may be expanded, washed, concentrated, and/or then frozen to allow time for testing, shipping and storage until a patient is ready to receive the infusion of cells.

Other aspects of the invention provide the use of a population of T cells as described herein for the manufacture of a medicament for the treatment of cancer, a population of T cells as described herein for the treatment of cancer, and a method of treatment of cancer comprising administering a population of T cells as described herein to an individual in need thereof.

The population of T cells may be autologous i.e. the T cells were originally obtained from the same individual to whom they are subsequently administered (i.e. the donor and recipient individual are the same).

The population of T cells may be allogeneic i.e. the T cells may be originally obtained from a different individual to the individual to whom they are subsequently administered (i.e. the donor and recipient individual are different). Allogeneic refers to a graft derived from a different animal of the same species.

The donor and recipient individuals may be HLA matched to avoid GVHD and other undesirable immune effects, such as rejection. Alternatively, the donor and recipient individuals may not be HLA matched, or HLA genes in the cells from the donor individual may be modified, for example by gene editing, to remove any HLA mismatch with the recipient.

A suitable population of T cells for administration to a recipient individual may be produced by a method comprising providing an initial population of cells, obtained from a donor individual, reprogramming the cells into iPSCs and differentiating the iPSCs into T cells that express an antigen receptor, such as an αβ TCR which binds specifically to cancer cells or antigen or peptide of antigen on cancer cells, optionally presented in complex with MHC, in the recipient individual.

Following administration of the T cells, the recipient individual may exhibit a T cell mediated immune response against cancer cells in the recipient individual. This may have a beneficial effect on the cancer condition in the individual.

As used herein, the terms “cancer,” “neoplasm,” and “tumour” are used interchangeably and, in either the singular or plural form, refer to cells that have undergone a malignant transformation that makes them pathological to the host organism.

Primary cancer cells can be readily distinguished from non-cancerous cells by well-established techniques, particularly histological examination. The definition of a cancer cell, as used herein, includes not only a primary cancer cell, but any cell derived from a cancer cell ancestor. This includes metastasized cancer cells, and in vitro cultures and cell lines derived from cancer cells. When referring to a type of cancer that normally manifests as a solid tumour, a “clinically detectable” tumour is one that is detectable on the basis of tumour mass; e.g., by procedures such as computed tomography (CT) scan, magnetic resonance imaging (MRI), X-ray, ultrasound or palpation on physical examination, and/or which is detectable because of the expression of one or more cancer-specific antigens in a sample obtainable from a patient. Cancer conditions may be characterised by the abnormal proliferation of malignant cancer cells and may include leukaemias, such as AML, CML, ALL and CLL, lymphomas, such as Hodgkin lymphoma, non-Hodgkin lymphoma and multiple myeloma, and solid cancers such as sarcomas, skin cancer, melanoma, bladder cancer, brain cancer, breast cancer, uterus cancer, ovary cancer, prostate cancer, lung cancer, colorectal cancer, cervical cancer, liver cancer, head and neck cancer, oesophageal cancer, pancreas cancer, renal cancer, adrenal cancer, stomach cancer, testicular cancer, cancer of the gall bladder and biliary tracts, thyroid cancer, thymus cancer, cancer of bone, and cerebral cancer, as well as cancer of unknown primary (CUP).

Cancer cells within an individual may be immunologically distinct from normal somatic cells in the individual (i.e. the cancerous tumour may be immunogenic). For example, the cancer cells may be capable of eliciting a systemic immune response in the individual against one or more antigens expressed by the cancer cells. The tumour antigens that elicit the immune response may be specific to cancer cells or may be shared by one or more normal cells in the individual.

The cancer cells of an individual suitable for treatment as described herein may express the antigen and/or may be of correct HLA type to bind the antigen receptor expressed by the T cells.

An individual suitable for treatment as described above may be a mammal. In preferred embodiments, the individual is a human. In other preferred embodiments, non-human mammals, especially mammals that are conventionally used as models for demonstrating therapeutic efficacy in humans (e.g. murine, primate, porcine, canine, or rabbit animals) may be employed.

In some embodiments, the individual may have minimal residual disease (MRD) after an initial cancer treatment.

An individual with cancer may display at least one identifiable sign, symptom, or laboratory finding that is sufficient to make a diagnosis of cancer in accordance with clinical standards known in the art. Examples of such clinical standards can be found in textbooks of medicine such as Harrison's Principles of Internal Medicine, 15th Ed., Fauci AS et al., eds., McGraw-Hill, New York, 2001. In some instances, a diagnosis of a cancer in an individual may include identification of a particular cell type (e.g. a cancer cell) in a sample of a body fluid or tissue obtained from the individual.

An anti-tumour effect is a biological effect which can be manifested by a reduction in the rate of tumour growth, decrease in tumour volume, a decrease in the number of tumour cells, a decrease in the number of metastases, an increase in life expectancy, or amelioration of various physiological symptoms associated with the cancerous condition. An “anti-tumour effect” can also be manifested by the ability of the peptides, polynucleotides, cells and antibodies, also T cells which may be obtained according to the methods of the present invention, as described herein in prevention of the occurrence of tumour in the first place Treatment may be any treatment and/or therapy, whether of a human or an animal (e.g. in veterinary applications), in which some desired therapeutic effect is achieved, for example, the inhibition or delay of the progress of the condition, and includes a reduction in the rate of progress, a halt in the rate of progress, amelioration of the condition, cure or remission (whether partial or total) of the condition, preventing, delaying, abating or arresting one or more symptoms and/or signs of the condition or prolonging survival of a subject or patient beyond that expected in the absence of treatment.

Treatment may also be prophylactic (i.e. prophylaxis). For example, an individual susceptible to or at risk of the occurrence or re-occurrence of cancer may be treated as described herein. Such treatment may prevent or delay the occurrence or re-occurrence of cancer in the individual.

In particular, treatment may include inhibiting cancer growth, including complete cancer remission, and/or inhibiting cancer metastasis. Cancer growth generally refers to any one of a number of indices that indicate change within the cancer to a more developed form. Thus, indices for measuring an inhibition of cancer growth include a decrease in cancer cell survival, a decrease in tumour volume or morphology (for example, as determined using computed tomographic (CT), sonography, or other imaging method), a delayed tumour growth, a destruction of tumour vasculature, improved performance in delayed hypersensitivity skin test, an increase in the activity of T cells, and a decrease in levels of tumour-specific antigens. Administration of T cells modified as described herein may improve the capacity of the individual to resist cancer growth, in particular growth of a cancer already present the subject and/or decrease the propensity for cancer growth in the individual.

The T cells or the pharmaceutical composition comprising the T cells may be administered to a subject by any convenient route of administration, whether systemically/ peripherally or at the site of desired action, including but not limited to; parenteral, for example, by infusion. Infusion involves the administration of the

T cells in a suitable composition through a needle or catheter. Typically, T cells are infused intravenously or subcutaneously, although the T cells may be infused via other non-oral routes, such as intramuscular injections and epidural routes. Suitable infusion techniques are known in the art and commonly used in therapy (see, e.g., Rosenberg et al., New Eng. J. of Med., 319:1676, 1988).

Typically, the number of cells administered is from about 10⁵ to about 10¹⁰ per Kg body weight, for example any of about 1, 2, 3, 4, 5, 6, 7, 8, or 9, ×10⁵, ×10⁶, ×10⁷, ×10⁸, ×10⁹, or ×10¹⁰ cells per individual, typically 2×10⁸ to 2×10¹⁰ cells per individual, typically over the course of 30 minutes, with treatment repeated as necessary, for example at intervals of days to weeks. It will be appreciated that appropriate dosages of the T cells, and compositions comprising the T cells, can vary from patient to patient. Determining the optimal dosage will generally involve the balancing of the level of therapeutic benefit against any risk or deleterious side effects of the treatments of the present invention. The selected dosage level will depend on a variety of factors including, but not limited to, the activity of the particular cells, cytokine release syndrome (CRS), the route of administration, the time of administration, the rate of loss or inactivation of the cells, the duration of the treatment, other drugs, compounds, and/or materials used in combination, and the age, sex, weight, condition, general health, and prior medical history of the patient. The dose of cells and the route of administration will ultimately be at the discretion of the physician, although generally the dosage will be to achieve local concentrations at the site of action which achieve the desired effect without causing substantial harmful or deleterious side-effects.

While the T cells may be administered alone, in some circumstances the T cells may be administered in combination with the target antigen, APCs displaying the target antigen, CD3/CD28 beads, IL-2 , IL-7 and/or IL15 to promote expansion in vivo of the population of T cells. Administration in combination may be by separate, simultaneous or sequential administration of the combined components.

The population of T cells may be administered in combination with one or more other therapies, such as cytokines e.g. IL-2, cytotoxic chemotherapy, radiation and immuno-oncology agents, including checkpoint inhibitors, such as anti-B7-H3, anti-B7-H4, anti-TIM3, anti-KIR, anti-LAG3, anti-PD-1, anti-PD-L1, and anti-CTLA4 antibodies. Administration in combination may be by separate, simultaneous or sequential administration of the combined components.

The one or more other therapies may be administered by any convenient means, preferably at a site which is separate from the site of administration of the T cells.

Administration of T cells can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the treating physician. Preferably, the T cells are administered in a single transfusion of any of 500 million, 1 billion, 2, 3, 4, 5, 6, 7, 8, 9, 10, 11, 12, 13, 14, 15 billion T cells for example at least 1 x 10⁹ T cells.

Other aspects of the invention provide kits and reagents for use in generating populations of haemogenic progenitor cells, for example HECs and HPCs, using the methods described above.

A kit for production of haemogenic progenitor cells may comprise;
a first mesoderm induction medium comprising activin,
a second mesoderm induction medium comprising activin, BMP and FGF,
a third mesoderm induction medium comprising activin, BMP, FGF, and a GSK3 inhibitor, and
a HE induction medium comprising VEGF and SCF.

The kit may further comprise an HPC induction medium comprising VEGF, SCF, Thrombopoietin (TPO), FIt3 ligand (FIt3L), IL-3, IL-6, IL-7, IL-11, IGF-1, BMP, FGF, Sonic hedgehog (SHH), erythropoietin (EPO), angiotensin II, and an angiotensin II type 1 receptor (AT1) antagonist.

Another aspect of the invention also provides the use of a set of culture media for the production of haemogenic progenitor cells, wherein the set of media comprises;
a first mesoderm induction medium comprising activin,
a second mesoderm induction medium comprising activin, BMP and FGF,
a third mesoderm induction medium comprising activin, BMP, FGF, and a GSK3 inhibitor, and
a HE induction medium comprising VEGF and SCF, and optionally
an HPC induction medium comprising VEGF, SCF, Thrombopoietin (TPO), FIt3 ligand (FIT3L), IL-3, IL-6, IL-7, IL-11, IGF-1, BMP, FGF, Sonic hedgehog (SHH), erythropoietin (EPO), angiotensin II, and an angiotensin II type 1 receptor (AT₁) antagonist.

Suitable media are described in more detail above.

Media may be supplemented with effective amounts of the differentiation factors set out above, as described elsewhere herein.

The one or more culture media may be formulated in deionized, distilled water. The one or more media will typically be sterilized prior to use to prevent contamination, e.g. by ultraviolet light, heating, irradiation or filtration. The one or more media may be frozen (e.g. at −20° C. or −80° C.) for storage or transport. The one or more media may contain one or more antibiotics to prevent contamination.

The one or more media may be a 1× formulation or a more concentrated formulation, e.g. a 2× to 250× concentrated medium formulation. In a 1x formulation each ingredient in the medium is at the concentration intended for cell culture, for example a concentration set out above. In a concentrated formulation one or more of the ingredients is present at a higher concentration than intended for cell culture. Concentrated culture media are well known in the art. Culture media can be concentrated using known methods e.g. salt precipitation or selective filtration. A concentrated medium may be diluted for use with water (preferably deionized and distilled) or any appropriate solution, e.g. an aqueous saline solution, an aqueous buffer or a culture medium.

The one or more media in the kit may be contained in hermetically-sealed vessels. Hermetically-sealed vessels may be preferred for transport or storage of the culture media, to prevent contamination. The vessel may be any suitable vessel, such as a flask, a plate, a bottle, a jar, a vial or a bag.

Other aspects and embodiments of the invention provide the aspects and embodiments described above with the term “comprising” replaced by the term “consisting of” and the aspects and embodiments described above with the term “comprising” replaced by the term “consisting essentially of”.

It is to be understood that the application discloses all combinations of any of the above aspects and embodiments described above with each other, unless the context demands otherwise. Similarly, the application discloses all combinations of the preferred and/or optional features either singly or together with any of the other aspects, unless the context demands otherwise.

Modifications of the above embodiments, further embodiments and modifications thereof will be apparent to the skilled person on reading this disclosure, and as such, these are within the scope of the present invention.

Unless defined otherwise, all technical and scientific terms used herein have the same meaning as commonly understood by one of ordinary skill in the art to which this invention belongs. Although any compositions and methods similar or equivalent to those described herein can be used in the practice or testing of the methods of the disclosure, exemplary compositions and methods are described herein. Any of the aspects and embodiments of the disclosure described herein may also be combined. For example, the subject matter of any dependent or independent claim disclosed herein may be multiply combined (e.g., one or more recitations from each dependent claim may be combined into a single claim based on the independent claim on which they depend).

Ranges provided herein include all values within a particular range described and values about an endpoint for a particular range. The figures and tables of the disclosure also describe ranges, and discrete values, which may constitute an element of any of the methods disclosed herein. Concentrations described herein are determined at ambient temperature and pressure. This may be, for example, the temperature and pressure at room temperature or in within a particular portion of a process stream. Preferably, concentrations are determined at a standard state of 20° C. and 1 bar of pressure. The term “about” means a value within two standard deviations of the mean for any particular measured value.

As used herein and in the claims, the singular forms “a,” “and,” and “the” include plural reference unless the context clearly dictates otherwise. Thus, for example, reference to “a peptide chain” is a reference to one or more peptide chains and includes equivalents thereof known to those skilled in the art.

All documents and sequence database entries mentioned in this specification are incorporated herein by reference in their entirety for all purposes.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

### EXPERIMENTAL

### Methods

### hiPSC Culture

iPSCs were routinely cultured in mTeSR1 (SCT) on Matrigel (BD Corning) using tissue culture plasticware, in 5% CO2, 5% 02 at 37° C. hiPSCs were harvested manually using an EasyPassage tool (Invitrogen) and cells seeded at 1:6 or 1:12 ratios, in media with Y27632 (R&D Systems) at 10 uM for the first 48h of culture. For differentiation, hiPSCs were passaged onto either Matrigel or vitronectin at low density cultures using 1:48 or 1:98 split ratio. Seeding density was about 1 colony per field of view, when viewed under x4 magnification on the microscope, at 24 h post seeding. hiPSCs were cultured in mTeSR1, TeSR2, or E8 flex (SCT), depending on the cell culture matrix used, for approx. 4-5 days until colonies were compacted and distinct cells were no longer visible.

### T cell Differentiation from Pluripotent Stem Cells

HiPSC maintenance medium (mTeSR1 or E8 flex) was removed, the cells were washed twice with DMEM/F12.

2 mL of StemPro34 PLUS (StemPro34 from Invitrogen; StemPro34 basal media, with supplement added and Penicillin Streptomycin (1% v/v: Invitrogen) and Glutamine (2mM: Invitrogen), Ascorbic Acid (50 μg/ml: Sigma Aldrich) and monothioglycerol (100 μM: Sigma Aldrich), further supplemented with 50 ng/mL of Activin was added and incubated for 4 hours. Volumes are dependent of culture flask size, typically at least 2 mls/9 cm², and 20 mls/150 cm².

After 4 hours, the medium was removed, and the cells were washed twice with DMEM/F12 to remove residual high concentration Activin A. The medium was replaced with 2 mL of StemPro34 PLUS supplemented with 5 ng/mL of Activin A, 10 ng/ml of BMP4 and 5 ng/ml of bFGF and incubated for 44 hours (Stage 1 media). The medium was then replaced with fresh Stage 1 media and supplemented with 10 μM CHIR-99021 and further cultured for 48 hours.

On Day 4, the medium was removed, and the cells were washed twice with DMEM/F12 to remove residual stage 1 cytokines. The medium was then replaced with StemPro34 PLUS supplemented with 100 ng/mL of SCF and 15 ng/ml of VEGF and incubated for 48 hours (Stage 2 media). The medium was then replenished with fresh Stage 2 media and the cells cultured for a further 48 hours.

The medium was then replaced by the Stage 3 medium shown in Table 1 and the cells were cultured for between 16-18 days, with 1:1 (v/v) feeding every 48 h. Typically this involved harvest of media and collection of cells in suspension by centrifugation (at 300 g, 10 min), and returning suspension cells to culture with fresh media (i.e. 20 ml for a T150 flask).

On approx. d16-18 depending on hiPSC line used, (confirmed separately by flow cytometry prior to day of harvest) we isolate CD34+ cells from resulting monolayers for onward culture. Here we routinely include hiPSCs lines designated ChiPSC31 (Takara), NIH2 (WT: a sub clone of MR1.1 from Lonza)) and sub-clones of NIH2: c3F3 and c1A12. CD34+ cells were harvested by sequential incubation with Accutase (SCT: for 30 min at 37° C.) and then Collagenase II (Invitrogen: 2 mg/ml) for 30 min at 37° C. Cell suspensions were collected and washed (x2 centrifugation at 300 g for 12 min in DMEM/F12), prior to CD34+ cell isolation via magnetic activated beads (MACS) isolation (Miltenyi: according to manufacturer's instructions).

Following MACS isolation of CD34+ cells, these were then cryopreserved at 2×10⁵ cells/vial in CS10 (SCT), firstly at −80° C. at slow rate freezing, then in liquid nitrogen for long term storage.

For continued lymphoid proliferation and differentiation, Stem Cell Technologies proprietary 2 stage (Lymphoid Proliferation /T cell Maturation) media was employed (according to manufacturer's instructions). Onward differentiation through lymphoid expansion medium and T cell maturation medium was successfully demonstrated resulting in double positive and single positive T cells and NK cells which could be further activated and expanded as described to produce functional T cells and NK cells which demonstrated mature T cell activity, for example cytokine production such as those cytokines for helping to suppress or regulate immune responses and cytotoxin production necessary for cell killing activity. In conclusion, the described methods achieve the production of haemogenic progenitor cells without use of serum or stromal co-culture, produced without the need of intermediate purification or isolation steps such that a single culture vessel may be used. The haemogenic progenitor cells have been shown to be onward differentiated to mature active T cells and NK cells.

TABLE 1

| | Final | |
|---|---|---|
| Reagent | Concentration | |
| StemPro34 | 1 L (2 bottles | |
| PLUS | required) | |
| VEGF | 15 | ng/mL |
| SCF | 100 | ng/mL |
| TPO | 30 | ng/mL |
| Flt3L | 25 | ng/mL |
| IL-3 | 25 | ng/mL |
| IL-6 | 10 | ng/mL |
| IL-7 | 10 | ng/mL |
| IL-11 | 5 | ng/mL |
| IGF-1 | 25 | ng/mL |
| BMP-4 | 10 | ng/mL |
| bFGF | 5 | ng/mL |
| SHH | 25 | ng/mL |
| EPO | 2 | U/mL |
| Angiotensin II | 10 | μg/mL |
| Losartan | 100 | μM |

TABLE 2
Antibodies used to phenotype T cells.

| Antibody | Volume/test (μl) |
|---|---|
| TCRαβ (IP26) PE (BioLegend: 306708) | 2.5 μl |
| TCRγδ (B1) APC (BioLegend: 331212) | 5 μl |
| CD5 (UCHT2) BV421 (BD: 562646) | 5 μl |
| CD7 (CD7-6B7) PerCP Cy5.5 (BioLegend: 343116) | 5 μl |
| CD45 (HI30) BUV395 (BD: 563792) | 5 μl |
| CD4 (OKT4) BV786 (BioLegend: 317442) | 5 μl |
| CD3 (SK7) AF488 (BioLegend: 344810) | 5 μl |
| CD8α (RPA-T8) PE-Cy7 (BD: 557746) | 5 μl |
| CD56 (NCAM16.2) BV605 (BD: 562780) | 5 μl |
| Ef506 BV510 (Invitrogen: 65-0866-14) | 1/100 dilution |

1. A method of producing a population of haemogenic progenitor cells comprising;
(i) differentiating a population of induced pluripotent stem cells (iPSCs) into mesoderm cells and;
(ii) differentiating the mesoderm cells to produce a population of haemogenic progenitor cells
wherein steps (i) and (ii) are performed without purification or isolation of cells in the population.

2. A method according to claim 1 wherein steps (i) and (ii) are performed in the absence of stromal cells or serum.

3. A method according to claim 1 wherein the haemogenic progenitor cells are haemogenic endothelial cells (HECs) or haematopoietic progenitor cells (HPCs)

4. A method according to any one of claims 1 to 3 wherein the iPSCs are differentiated into mesoderm cells by culturing the population of iPSCs under suitable conditions to promote mesodermal differentiation.

5. A method according to any one of claims 1 to 4 wherein the iPSCs are cultured sequentially in first, second and third mesoderm induction media to induce differentiation into mesoderm cells.

6. A method according to claim 5 wherein the first mesoderm induction medium stimulates SMAD2 and SMAD3 mediated signalling pathways. A method according to claim 6 wherein the first mesoderm induction medium comprises activin.

8. A method according to claim 6 or claim 7 wherein the first mesoderm induction medium consists of a chemically defined nutrient medium supplemented with one or more differentiation factors, wherein the one or more differentiation factors consist of activin.

9. A method according to any one of claims 4 to 7 wherein the second mesoderm induction medium (i) stimulates SMAD1, SMAD2, SMAD3, SMAD5 and SMAD9 mediated signalling pathways and (ii) has fibroblast growth factor (FGF) activity.

10. A method according to claim 9 wherein the second mesoderm induction medium comprises activin, BMP, and FGF.

11. A method according to claim 9 or claim 10 wherein the second mesoderm induction medium consists of a chemically defined nutrient medium supplemented with one or more differentiation factors, wherein the one or more differentiation factors consist of activin, BMP, and FGF.

12. A method according to any one of claims 5 to 11 wherein the third mesoderm induction medium (i) stimulates SMAD1, SMAD2, SMAD3, SMAD5 and SMAD9 mediated signalling pathways (ii) has fibroblast growth factor (FGF) activity and (iii) inhibits glycogen synthase kinase 3β.

13. A method according to claim 12 wherein the third mesoderm induction medium comprises activin, BMP, FGF, and a GSK3 inhibitor.

14. A method according to claim 13 wherein the third mesoderm induction medium consists of a chemically defined nutrient medium supplemented with one or more differentiation factors, wherein the one or more differentiation factors consist of activin, BMP, FGF, and a GSK3 inhibitor.

15. A method according to any one of claims 5 to 14 wherein the mesoderm cells express one or more mesoderm markers selected from Brachyury Goosecoid, MixI1, KDR, FoxA2, GATA6, and PDGFαR.

16. A method according to any one of claims 5 to 16 wherein mesoderm cells in the population are not purified following culture in the first, second and third mesoderm induction media.

17. A method according to any one of the preceding claims wherein the mesoderm cells are differentiated into HECs by culturing the population of mesoderm cells under suitable conditions to promote haemogenic endothelial (HE) differentiation.

18. A method according to any one of the preceding claims wherein the mesoderm cells are cultured in an HE induction medium to induce differentiation into HECs.

19. A method according to claim 18 wherein the HE induction medium (i) stimulates cKIT receptor (CD117; KIT receptor tyrosine kinase) mediated signalling pathways and/or (ii) stimulates VEGFR mediated signalling pathways.

20. A method according to claim 19 wherein the HE induction medium comprises SCF and/or VEGF.

21. A method according to claim 20 wherein the HE induction medium consists of a chemically defined nutrient medium supplemented with one or more differentiation factors, wherein the one or more differentiation factors consist of SCF and VEGF.

22. A method according to any of claims 18 to 21 wherein the HECs display a CD34+ phenotype.

23. A method according to any of claims 18 to 22 wherein HECs in the population are not purified following culture in the HE induction medium.

24. A method according to any one of the preceding claims wherein the HECs are differentiated into HPCs by culturing the population of HECs under suitable conditions to promote haematopoietic differentiation.

25. A method according to any one of the preceding claims wherein the HECs are cultured in an haematopoietic induction medium to induce differentiation into HPCs.

26. A method according to claim 25 wherein the haematopoietic induction medium stimulates (i) stimulates cKIT receptor (CD117) mediated signalling pathways, (ii) VEGFR mediated signalling pathways, (iii) MPL (CD110) mediated signalling pathways (iv) FLT3 mediated signalling pathways (v) IGF1R mediated signalling pathways (vi) SMAD1, 5 and 9 mediated signalling pathways (vii) Hedgehog signalling pathways (viii) EpoR mediated signalling pathway and (ix) AGTR2 mediated signalling pathways; inhibits the AGTR1 signalling pathway and displays IL and FGF activity.

27. A method according to claim 26 wherein the haematopoietic induction medium comprises VEGF, SCF, Thrombopoietin (TPO), FIt3 ligand (FIt3L), IL-3, IL-6, IL-7, IL-11, IGF-1, BMP, FGF, Sonic hedgehog (SHH), erythropoietin (EPO), angiotensin II, and an angiotensin II type 1 receptor (AT₁) antagonist.

28. A method according to claim 27 wherein the haematopoietic induction medium consists of a chemically defined nutrient medium supplemented with one or more differentiation factors, wherein the one or more differentiation factors consist of VEGF, SCF, Thrombopoietin (TPO), FIt3 ligand (FIt3L), IL-3, IL-6, IL-7, IL-11, IGF-1, BMP, FGF, Sonic hedgehog (SHH), erythropoietin (EPO), angiotensin II, and an angiotensin II type 1 receptor (AT₁) antagonist.

29. A method according to any one of claims 24 to 28 wherein the HPCs display a CD34+ CD45+ phenotype.

30. A method according to any one of claims 24 to 29 comprising purifying the population of HPCs.

31. A method according to any one of the preceding claims wherein the haemogenic progenitor cells areHPCs and the method further comprises differentiating the population of HPCs into progenitor T cells.

32. A method according to claim 31 wherein the HPCs are differentiated by a method comprising culturing the population of HPCs in a lymphoid expansion medium to produce the progenitor T cells.

33. A method according to claim 31 or claim 32 wherein the progenitor T cells have a CD5+ CD7+ phenotype.

34. A method according to any one of claims 31 to 33 further comprising maturing the progenitor T cells to produce a population of T cells.

35. A method according to claim 34 wherein the progenitor T cells are matured by a method comprising culturing the population of progenitor T cells in a T cell maturation medium to produce the T cells.

36. A method according to claim 34 or claim 35 wherein the T cells have a CD8+ CD4+ phenotype.

37. A method according to any one of claims 34 to 36 comprising activating and expanding the T cells to produce a population of T cells have a CD8+ single positive phenotype or a CD4+ single positive phenotype.

38. A method according to any one of claims 34 to 37 wherein the T cells specifically bind to cells expressing a target antigen.

39. A method according to claim 38 wherein the target antigen is a tumour antigen.

40. A method according to claim 39 wherein the T cells specifically bind to cancer cells expressing the tumour antigen.

41. A method according to any one of the preceding claims wherein the iPSCs are derived from T cells obtained from a donor individual.

42. A method according to claim 41 wherein the T cells obtained from the donor individual are specific for the target antigen.

43. A method according to claim 41 or 42 wherein the T cells obtained from the donor individual are tumour-infiltrating lymphocytes (TILs).

44. A method according to any one of claims 1 to 40 wherein the method further comprises introducing heterologous nucleic acid encoding an antigen receptor into the iPSCs, HPCs or progenitor T cells.

45. A method according to claim 44 wherein the heterologous nucleic acid encoding the antigen receptor is comprised in an expression vector.

46. A method according to claim 45 wherein the expression vector is a lentiviral vector or adeno-associated viral (AAV) vector.

47. A method according to claim 44 or 45 wherein the heterologous nucleic acid is incorporated into the genome of the iPSCs, HECs, haemogenic progenitor cells, or progenitor T cells using a gene editing system.

48. A method according to claim 47 wherein the gene editing system is CRISPR/Cas9 or AAV.

49. A method according to any one of claims 44 to 48 wherein the antigen receptor is a TCR.

50. A method according to claim 49 wherein the TCR is TCR is an affinity enhanced TCR.

51. A method according to claim 49 wherein the TCR is TCR is a non-MHC restricted TCR.

52. A method according to one of claims 49 to 51 wherein the TCR binds specifically to an MHC displaying a peptide fragment of a target antigen expressed by cells or specifically binds to a target antigen or peptide thereof expressed by cells independently of MHC presentation.

53. A method according to claim 52 wherein the TCR binds specifically to an MHC displaying a peptide fragment of a tumour antigen expressed by the cancer cells or binds specifically to a tumour antigen or peptide fragment thereof expressed by cancer cells independently of MHC presentation.

54. A method according to any one of claims 44 to 48 wherein the antigen receptor is a chimeric antigen receptor (CAR) or NKCR.

55. A method according to claim 54 wherein the CAR or NKCR binds specifically to a target antigen expressed by cells.

56. A method according to claim 55 wherein the CAR or NKCR binds specifically to an MHC displaying a peptide fragment of a tumour antigen expressed by cancer cells.
